(19) United States
(12) Patent Application Publication
(10) Pub. No.: US 2007/0161550 A1
(43) Pub. Date: Jul. 12, 2007

(54) Antiviral compositions which inhibit paramyxovirus infection

(75) Inventors: Gerald E. Hancock, New City, NY (US); Paul William Tebbey, Pottstown, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/585,046

(22) PCT Filed: Dec. 28, 2004
(86) PCT No.: PCT/US04/43930

§ 371 Date: Jun. 29, 2006

Related U.S. Application Data
(60) Provisional application No. 60/533,445, filed on Dec. 30, 2003.

(51) Int. Cl. A61K 38/16; A61K 38/10

(52) U.S. Cl. 514/12; 514/14

(57) ABSTRACT

The invention is directed to an antiviral for administration to a mammalian host (e.g., a human) susceptible to paramyxovirus infection, particularly respiratory syncytial virus (RSV) infection. In certain embodiments, an antiviral molecule of the invention is a polypeptide, a chemokine polypeptide, a chemokine polypeptide fragment, an organic small molecule or a peptide mimetic, wherein the antiviral molecule inhibits or prevents paramyxovirus infection of a mammalian cell.

### FIELD OF THE INVENTION

The present invention generally relates to the fields of microbiology, virology, infectious disease and immunology. More particularly, the invention relates to polypeptides, polypeptide fragments and small organic molecules which inhibit or prevent infection by a virus of the Family Paramyxoviridae (paramyxovirus) in a mammal.

### BACKGROUND OF THE INVENTION

Respiratory syncytial virus (RSV) is a non-segmented negative strand RNA virus and major cause of lower respiratory tract (LRT) disease in young infants, patients with underlying disease or immunological abnormalities, and aged adults (Domachowske and Rosenberg, 1999; Hall, 2001). In fact, sixty-five million RSV infections occur globally every year, resulting in 160,000 deaths (Robbins and Freeman, 1988). In the United States alone, 100,000 children are hospitalized annually with severe cases of pneumonia and bronchiolitis resulting from an RSV infection (Glezen et al., 1986; Katz, 1985). Inpatient and ambulatory care for children with RSV infections in the U.S. was estimated in 1992 to cost in excess of $340 million per year (Wertz and Sullender, 1992).

Currently a licensed vaccine to prevent human disease is not available. Thus, immunologically naïve infants born during seasonal epidemics must rely on maternally derived antibodies to prevent severe LRT disease caused by RSV. For high-risk infants this may be problematic due to the immune status of the mother and the finite half-life of maternally derived antibody. Presently, two biologic medicines, IVIG and a humanized monoclonal antibody, palivizumab (Synagis®), are approved to prevent infection in high-risk infants (Hall, 2001; Krilov, 2002). Although the benefits of immunoprophylaxis are demonstrable, the costs are significant. In addition to the biologic medicines described above, there is also one pharmaceutical agent (ribavirin) licensed to treat acute respiratory tract disease caused by RSV (Torrence and Powel, 2002). However, ribavirin is a teratogen and poses safety-risks to hospital staff, parents, and other relatives of childbearing age; and as such the benefits of ribavirin are controversial (Hall, 2001; Krilov, 2002). Thus, in the absence of a licensed vaccine, there is currently a great need for novel pharmaceutical and/or biologic compounds with improved effectiveness and an increased safety profile to prevent LRT disease caused by paramyxoviruses such as RSV.

### SUMMARY OF THE INVENTION

The present invention broadly relates to antiviral compositions which inhibit or prevent infection in a mammal. More particularly, the invention relates to novel antiviral molecules and pharmaceutical compositions thereof, which inhibit or prevent infection by a virus of the Family Paramyxoviridae (paramyxovirus) in a mammalian cell.

Thus, in certain embodiments, the invention is directed to an antiviral composition comprising a CCL5 polypeptide, wherein the CCL5 polypeptide inhibits infection by a virus of the Family Paramyxoviridae (paramyxovirus) in a mammalian subject. In one particular embodiment, the paramyxovirus is a respiratory syncytial virus (RSV). In another embodiment, the CCL5 polypeptide inhibits RSV infection by blocking the interaction between an RSV fusion (F) protein and a mammalian epithelial cell. In certain embodiments, the CCL5 polypeptide is a synthetic CCL5 polypeptide or a recombinantly expressed CCL5 polypeptide. In certain other embodiments, the CCL5 polypeptide is biologically inactive as a chemokine in a mammalian subject. In a particular embodiment, the mammalian subject is a human. In other embodiments, the mammalian subject is a domesticated non-human mammal selected from the group consisting of a cow, a horse, a pig, a dog, a cat, a goat and a sheep.

In another embodiment, the CCL5 polypeptide comprises an amino acid sequence of SEQ ID NO:1. In certain embodiments, the CCL5 polypeptide is an NH₂-terminus modified CCL5 polypeptide. In one particular embodiment, the NH₂-terminus modified CCL5 polypeptide is selected from the group consisting of an aminooxypentane-CCL5 (AOP-CCL5), a Met-CCL5, a N-nonanoyl-CCL5 (NNY-CCL5), a Δ1-2 truncated CCL5 and a Δ1-8 truncated CCL5. In other embodiments, an antiviral composition of the invention further comprises one or more CCL5 peptide fragments, wherein the fragments comprise about 10 to 20 contiguous amino acids of the CCL5 polypeptide of SEQ ID NO:1. In particular embodiments, the one or more CCL5 peptide fragments are selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18. In other embodiments, a CCL5 peptide fragment comprises an amino acid sequence of SEQ ID NO:2. In certain embodiments, the peptide fragment of SEQ ID NO:2 is further defined as an NH₂-terminal peptide of SEQ ID NO:1. In certain other embodiments, the CCL5 polypeptide is further defined as a human CCL5 polypeptide.

In yet other embodiments, an antiviral composition of the invention further comprises a peptide mimetic of the NH₂-terminus of the CCL5 polypeptide of SEQ ID NO:1. In one particular embodiment, the peptide mimetic of the NH₂-terminus of the CCL5 polypeptide is a retroinverted CCL5 polypeptide comprising an amino acid sequence of SEQ ID NO:19, SEQ ID NO:20 or SEQ ID NO:21.

In certain other embodiments, an antiviral composition of the invention further comprises an organic molecule which binds a CCR3 chemokine receptor. In certain of these embodiments, the organic molecule is a CCR3 receptor antagonist. In one particular embodiment, the organic molecule comprises one or more of the following chemical structures:

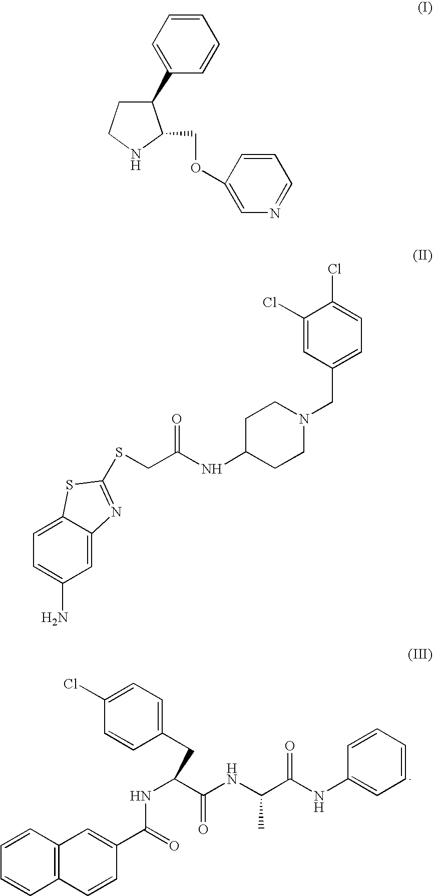

In certain other embodiments, an antiviral composition of the invention is administered to a mammalian subject by intranasal administration or parenteral administration.

In other embodiments, an antiviral composition of the invention further comprises an organic molecule which is a CCR1 antagonist or a CCR5 antagonist. In certain embodiments, an organic molecule which is a CCR1 antagonist comprises one or more of the following chemical structures:

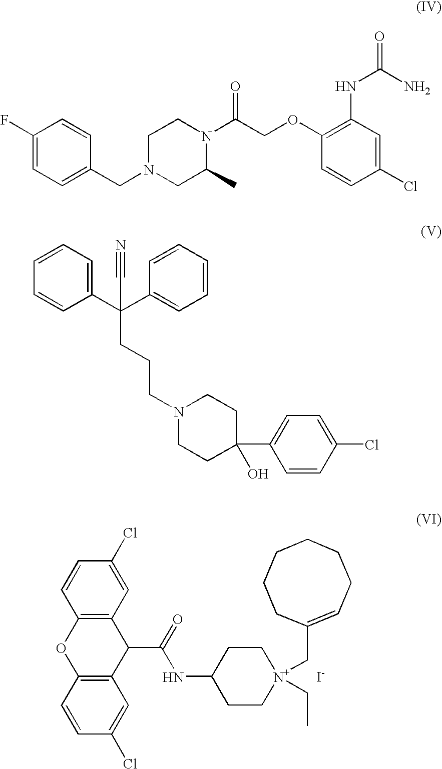

In certain embodiments, an organic molecule which is a CCR5 antagonist comprises one or more of the following chemical structures:

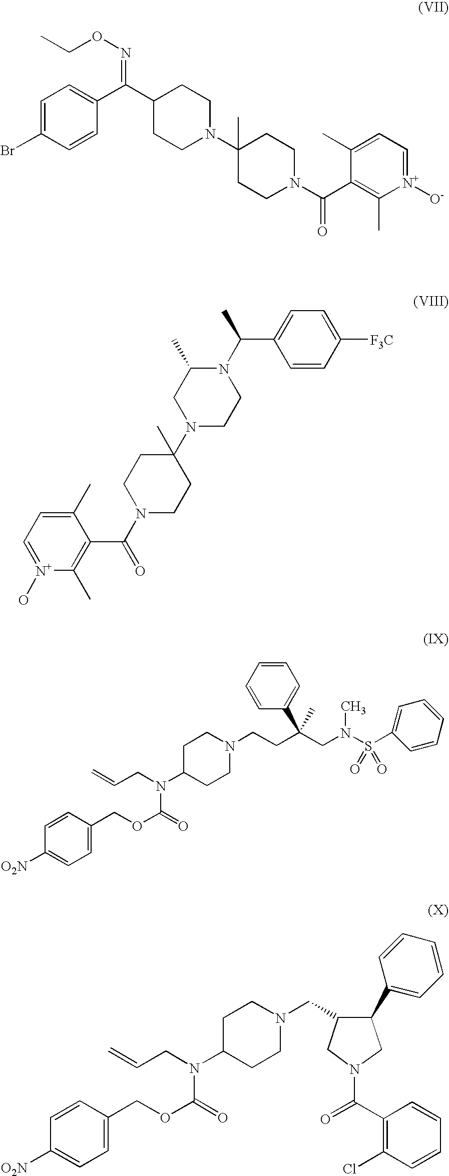

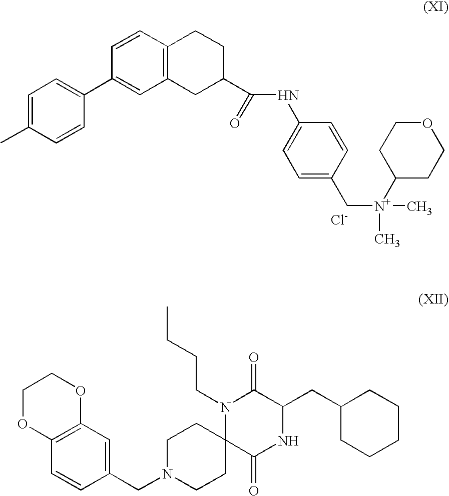

In other embodiments, the invention is directed to a recombinant expression vector comprising a polynucleotide sequence encoding the CCL5 polypeptide. In still other embodiments, the invention is directed to a host cell transfected, transformed or infected with the vector comprising a polynucleotide sequence encoding the CCL5 polypeptide.

In another embodiment, the invention is directed to an antiviral composition comprising an NH₂-terminal peptide fragment of a CCL5 polypeptide, wherein the fragment comprises about 10 to 20 contiguous amino acids of the NH₂-terminus of a CCL5 polypeptide, wherein the fragment inhibits infection by a virus of the Family Paramyxoviridae (paramyxovirus) in a mammalian subject. In one particular embodiment, the paramyxovirus is RSV. In another embodiment, the CCL5 polypeptide comprises an amino acid sequence of SEQ ID NO:1. In other embodiments, the NH₂-terminal peptide fragment comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18. In certain embodiments, the NH₂-terminal peptide fragment comprises an amino acid sequence of SEQ ID NO:2. In certain other embodiments, the composition is biologically inactive as a chemokine in a mammalian subject. In still other embodiments, the antiviral composition is administered to a mammalian subject by intranasal administration or parenteral administration. In yet other embodiments, the NH₂-terminal CCL5 peptide fragment inhibits RSV infection by blocking the interaction between an RSV fusion (F) protein and a mammalian epithelial cell. In certain other embodiments, the antiviral composition further comprises one or more NH₂-terminus modified CCL5 polypeptides selected from the group consisting of an aminooxypentane-CCL5 (AOP-CCL5), a Met-CCL5, a N^{α}-nonanoyl-CCL5 (NNY-CCL5), a Δ1-2 truncated CCL5 and a Δ1-8 truncated CCL5. In one embodiment, an antiviral composition further comprises a peptide mimetic of the NH₂-terminus of the CCL5 polypeptide of SEQ ID NO:1. In one particular embodiment, the peptide mimetic of the NH₂-terminus of the CCL5 polypeptide is a retroinverted CCL5 polypeptide comprising an amino acid sequence of SEQ ID NO:19, SEQ ID NO:20 or SEQ ID NO:21. In another embodiment, an antiviral composition further comprises an organic molecule which is an antagonist of a CCR1 receptor, a CCR3 receptor or a CCR5 receptor. In one particular embodiment, an antagonist of a CCR1 receptor, a CCR3 receptor or a CCR5 receptor comprises a chemical structure as represented by formulae I-XII.

In certain other embodiments, the invention is directed to a recombinant expression vector comprising a polynucleotide sequence encoding an NH₂-terminal CCL5 peptide fragment. In certain other embodiments, the invention is directed to a host cell transfected, transformed or infected with the vector a comprising a polynucleotide sequence encoding an NH₂-terminal CCL5 peptide fragment.

In certain other embodiments, the invention is directed to an organic small molecule mimetic which is designed by computer based molecular modeling using the atomic X, Y, Z coordinates of the first fifteen CCL5 NH₂-terminal amino acids of SEQ ID NO:1, wherein the X, Y, Z, coordinates are found in a Brookhaven Protein Data Bank file selected from the group consisting of 1 RTN, 1 RTO, 1 EQT and 1 B3A. In another embodiment, the invention is directed to an antiviral composition comprising an organic molecule designed by computer based molecular modeling described above.

In certain other embodiments, the invention is directed to a peptide mimetic of the NH₂-terminus of a CCL5 polypeptide, wherein the peptide mimetic inhibits infection by a virus of the Family Paramyxoviridae (paramyxovirus) in a mammalian subject. In certain embodiments, the mimetic is designed by computer based molecular modeling using the atomic X, Y, Z coordinates of the first fifteen CCL5 NH₂-terminal amino acids of SEQ ID NO:1, wherein the X, Y, Z, coordinates are comprised in a Brookhaven Protein Data Bank file selected from the group consisting of 1RTN, 1RTO, 1EQT and 1B3A. In one particular embodiment, the peptide mimetic is a reverse turn mimetic. In certain embodiments, the reverse turn mimetic is a β-turn mimetic, a monocyclic p-turn mimetic, a bicyclic β-turn mimetic, a γ-turn mimetic or a monocyclic γ-turn mimetic. In certain other embodiments, the peptide mimetic is comprised in an antiviral composition.

In certain other embodiments, the invention is directed to a method for preventing or inhibiting infection by a virus of the Family Paramyxoviridae (paramyxovirus) in a mammalian host, the method comprising administering to the host a pharmaceutically effective amount of an antiviral composition of the invention.

Other features and advantages of the invention will be apparent from the following detailed description, from the preferred embodiments thereof, and from the claims.

### BRIEF DESCRIPTION OF THE FIGURES

FIG. 1 shows that CCL5 inhibits RSV infection at the epithelial surface. Hep-2 cell monolayers were pre-treated (one hour before infection) with the indicated doses of recombinant rCCL5 (circles) or met-CCL5 (squares) before infection with RSV A2. Three days thereafter, the plaques were enumerated and presented as percent infectivity (±1 standard deviation) relative to control wells (100% infectivity) incubated with virus in medium alone and not exposed to chemokine.

FIG. 2 shows the expression of CCR1, CCR3 and CCR5 on HEp-2 and A549 cells. HEp-2 and A549 cell monolayers were gently removed from tissue culture flasks using a cell scraper. The cells were stained with anti-human CCR1 or anti-CCR3 mAb conjugated to phycoerythrin, or anti-CCR5 conjugated to FITC (FL2-H, x-axis, solid lines). Relative cell numbers (counts) are shown on the y-axis.

FIG. 3 shows the binding of overlapping synthetic peptides of CCL5 to Hep-2 cell monolayers. Viable Hep-2 cell monolayers were incubated (4° C.) with increasing concentrations (0.0039-4.0 μg/ml) of the denoted biotinylated peptide. After rinsing, monolayers were fixed in methanol and binding of peptide was visualized by ELISA at OD450-550.

FIGS. 4A and 4B show hydropathy plots of the CCL5 polypeptide versus the CCL3 polypeptide. FIG. 4A is a hydropathy plot of CCL5 (amino acids 5 to 68 of SEQ ID NO:1) aligned with CCL3 (amino acids 5 to 69 of SEQ ID NO:22). FIG. 4B is a hydropathy plot of CCL5 (amino acids 5 to 31 of SEQ ID NO:1) aligned with CCL3 (amino acids 5 to 31 of SEQ ID NO:22). The hydropathy scale on the y-axis was generated using amino acid hydropathy values of Kyte and Doolittle (1982), with a nine amino acid moving average window. The negative hydropathy values on the scale represent a hydrophilic environment and positive numbers on the scale represent a hydrophobic environment.

### DETAILED DESCRIPTION OF THE INVENTION

The invention described hereinafter addresses a need in the art for effective antiviral molecules for administering to a mammalian host (e.g., a human) susceptible to paramyxovirus infection, particularly respiratory syncytial virus (RSV) infection. Thus, in certain embodiments, the invention is directed to novel antiviral molecules and pharmaceutical compositions thereof. As defined hereinafter, an “antiviral molecule” of the invention is a “polypeptide”, a “chemokine polypeptide”, a chemokine “polypeptide fragment” (hereinafter, a “peptide fragment”), an “organic small molecule” (or “small molecule mimetic”) or a “peptide mimetic” (or “peptidomimetic”), wherein the antiviral molecule inhibits or prevents paramyxovirus infection of a mammalian cell.

Chemokines are small molecular weight cytokines that play a central role in directing the movement of cells towards a site of injury or infection (Baggiolini, 2001). For example, the chemokine CCL5 (also known as “RANTES”; which is an acronym for regulated on activation, normal T-cell expressed and secreted) is believed to play a major role in recruiting leukocytes to areas of tissue damage caused by RSV replication (Appay and Rowland-Jones, 2001; Moser and Loetscher, 2001). Chemokines are divided into subfamilies based upon number and spacing of conserved cysteine motifs termed C, CC, CXC, and CX3C. It has previously been established that infection with RSV induces gene expression and secretion of chemokines in airway epithelial cells (Harrison, 1999; Noah et al., 2002; Zhang, 2001). In addition, certain chemokine polypeptides of the CC family have been shown to possess potent antiviral properties against human immunodeficiency virus type 1 (HIV-1) (Lusso, 2002; Lehner, 2002; Proudfoot et al., 2003). For example, HIV-1 enters T-cells and macrophages by binding CCR5 as a primary co-receptor. The chemokine polypeptides CCL5 (RANTES), CCL3 (MIP-1α) and CCL4 (MIP-1β) are inhibitory ligands which block CCR5 as an HIV-1 co-receptor and thereby prevent HIV-1 infection.

The present invention shows for the first time that recombinant CCL5 (rCCL5) polypeptide, N-terminally modified CCL5 polypeptide and N-terminal CCL5 peptide fragments (a) inhibit infection of human epithelial cells with RSV (Example 2), (b) block the interaction between epithelial cells and fusion (F) protein of RSV (Example 3) and (c) inhibit RSV infection in vivo (Example 4).

In contrast to published reports of chemokine inhibition of HIV-1 infection (e.g., chemokines CCL3, CCL4 and CCL5) (Lusso, 2002; Lehner, 2002; Proudfoot et al., 2003), the data of the present invention demonstrate that chemokine inhibition of RSV infection only occurs with CCL5, but not with other CC inhibitors such as CCL3 (MIP-1α) or CCL4 (MIP-1β) (see Example 2), suggesting that RSV may utilize a receptor other than CCR5 (i.e., in contrast to HIV-1).

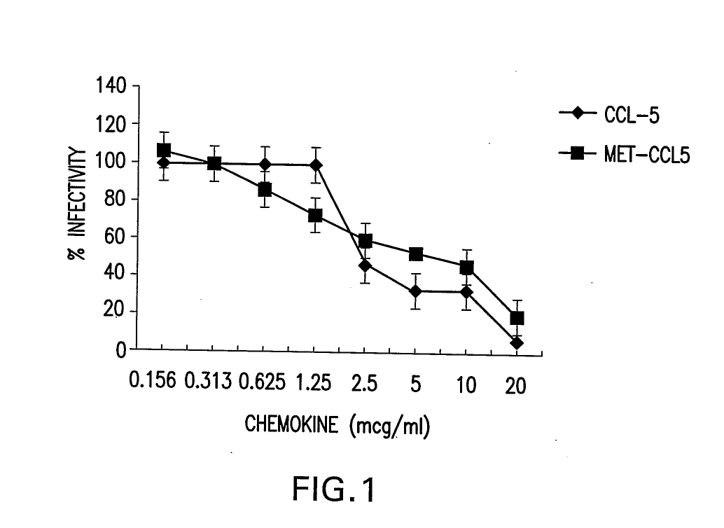

Human epithelial cells were examined by flow cytometry for receptors (CCR1, CCR3 and CCR5) known to bind CCL5. The results demonstrated that CCR3 (but not CCR1 or CCR5) was expressed on the surface of HEp-2 and A549 epithelial cells (see, Example 3 and FIG. 1), suggesting that CCL5 blocks the interaction(s) between RSV and CCR3 on the epithelial cell surface. Additional recombinant CC chemokines known to bind CCR3 (Baggiolini, 2001) were tested to determine whether they too reduced RSV infectivity. Prior treatment of HEp-2 cell monolayers with increasing amounts of recombinant CCL11 (eotaxin), CCL8 (MCP-2) or CCL15 (MIP-1δ) did not impair RSV infectivity (Table 10 and Table 12). In addition, pre-incubation of HEp-2 cell monolayers in the presence of poly or monoclonal anti-chemokine receptor antibodies directed against CCR1, CCR3 and/or CCR5 did not reduce RSV infectivity (data not shown).

It has previously been demonstrated in vitro, using mutant RSV strains lacking the SH (small hydrophobic) protein and/or G (attachment) protein, that the F (fusion) protein alone is sufficient to mediate RSV attachment (Karron et al., 1997). Thus, CCL5 inhibition was further investigated by examining the ability of rCCL5 to inhibit infection by RSV strains deficient in the G protein and/or the SH protein. A series of studies were performed using genetically modified RSV strains deleted of the SH protein (RSVΔSH) or with the C-terminal ectodomain of G protein truncated at amino acid 118 (RSVΔ118). These studies indicated that pre-treatment with 10 μg/ml of rCCL5 or Met-CCL5 reduced the infectivity of RSVΔ118 and RSVΔSH viruses relative to control cells cultured with virus in medium alone (Example 3, Table 15). Prior treatment with rCCL5 (10 μg/ml) also reduced infection by mutant cp32/D1 (lacking both SH and G proteins) and parent B1 strains of RSV (Example 3, Table 15). Thus, rCCL5 inhibited infection by RSV strains deficient in G and/or SH proteins.

To determine which region of CCL5 inhibits RSV infection, a series of nine CCL5 peptide fragments (15-mers, overlapping by seven amino acids) representing all sixty-eight amino acids of SEQ ID NO:1 were synthesized (Example 4 and Table 2) and tested in an in vivo mouse model for infection. Peptide 1 (SEQ ID NO:2), representing the first fifteen amino acid residues of the CCL5 NH2-terminus, was the most inhibitory peptide in vivo when administered simultaneously with, or prior to, RSV infection (Example 4, Table 16 and Table 17)

Taken together, the data of the present invention indicate a novel mechanism of CCL5 inhibition, wherein CCL5 blocks the interaction(s) that occur between the F protein in the RSV envelope and the epithelial cell surface. In addition, CCL5 peptide fragments, representing the NH2-terminal portion of CCL5, have been identified which inhibit RSV infection.

Thus, in certain embodiments, an antiviral molecule of the invention is a CCL5 polypeptide, an NH₂-terminally modified CCL5 polypeptide, an NH₂-terminal CCL5 peptide fragment, a modified NH₂-terminal CCL5 peptide fragment, a peptide mimetic designed to mimic an NH₂-terminal portion of the CCL5 polypeptide or a small molecule designed to mimic an NH₂-terminal portion of the CCL5 polypeptide, wherein the antiviral molecule inhibits or prevents paramyxovirus infection of a mammalian cell, particularly an epithelial cell. As defined hereinafter, a “paramyxovirus” encompasses virus of the Family Paramyxoviridae, including, but not limited to, RSV, Parainfluenza viruses (PIV) types 1-4, Measles virus, Mumps virus, human Metapneumovirus, Nipah virus, Hendra virus, Rinderpest virus and Canine distemper virus.

### A. ANTIVIRAL MOLECULES

As stated supra, an antiviral molecule of the invention is a polypeptide, a peptide fragment, an organic small molecule or a peptide mimetic which inhibits or prevents paramyxovirus infection of a mammalian cell. More particularly, an antiviral molecule is a polypeptide, peptide fragment, organic small molecule or peptide mimetic which blocks, inhibits or prevents the interaction(s) between the F protein of a paramyxovirus and an epithelial cell surface receptor, thereby preventing the paramyxovirus from entering the cell. Antiviral polypeptides (and fragments thereof) and antiviral peptide mimetics/small molecule mimetics of the invention are set forth below in Sections A.1 and A.2, respectively.

1. CCL5 Polypeptides and Fragments Thereof

In certain embodiments, an antiviral molecule of the invention is a CCL5 polypeptide, a chemically modified CCL5 polypeptide or a genetically modified CCL5 polypeptide. In one particular embodiment, a CCL5 polypeptide is modified at its NH₂-terminus. In other embodiments, an antiviral molecule of the invention is an NH₂-terminal CCL5 peptide fragment, a chemically modified NH₂-terminal CCL5 peptide fragment or a genetically modified NH₂-terminal CCL5 peptide fragment, wherein the peptide fragment comprises an NH₂-terminal portion of a CCL5 polypeptide which inhibits paramyxovirus infection of a mammalian cell.

As defined hereinafter, a full length CCL5 polypeptide has a molecular weight of about 7.8 kDa and comprises an amino acid sequence of SEQ ID NO:1. In certain embodiments, a full length CCL5 polypeptide of SEQ ID NO:1 is a synthetic CCL5 polypeptide or a recombinantly expressed CCL5 polypeptide. A full length CCL5 polypeptide of the invention comprises an amino acid sequence that has at least 95% identity to the amino acid sequence of SEQ ID NO:1, wherein the polypeptide inhibits or prevents paramyxovirus infection of a mammalian cell. Thus, a CCL5 polypeptide encompasses a polypeptide that comprises (a) the amino acid sequence shown in SEQ ID NO:1, (b) naturally occurring allelic variants of the polypeptide of SEQ ID NO:1, (c) polypeptides isolated from organisms other than human (e.g., orthologues of CCL5 polypeptides) and (d) NH₂-terminally modified CCL5 polypeptides of SEQ ID NO:1.

An allelic variant of a CCL5 polypeptide according to the present invention encompasses a polypeptide (1) which is isolated from a human cell and (2) that contains substantial homology to a human CCL5 polypeptide of SEQ ID NO:1. Allelic variants of a CCL5 polypeptide are naturally occurring amino acid sequence variants of a CCL5 polypeptide that maintain the ability to inhibit or prevent paramyxovirus infection of a mammalian cell. Thus, an allelic variant of a CCL5 polypeptide is defined as a “functional variant”. Functional variants contain only conservative substitutions of one or more amino acids of SEQ ID NO:1, or substitution, deletion or insertion of non-critical residues in non-critical regions of the polypeptide. For example, an NH₂-terminal CCL5 peptide fragment (SEQ ID NO:2) was observed to inhibit paramyxovirus infection of mammalian epithelial cells (Example 4). In addition, structural and functional studies of CCL5 inhibition of HIV-1 infection further suggest that amino acid residues Phe12, Tyr14 and Ile15 of CCL5 are critical for anti-HIV-1 activity (Nardese et al., 2001). Thus, in certain preferred embodiments, a allelic variant of a full length CCL5 polypeptide comprises a polypeptide having substantial homology to a human CCL5 polypeptide of SEQ ID NO:1, wherein the polypeptide does not comprise a substitution or deletion of amino acids Phe12, Tyr14 and Ile15.

The present invention further provides non-human orthologues of CCL5 polypeptides. Orthologues of CCL5 polypeptides are polypeptides that are isolated from mammalian, non-human organisms and possess antiviral properties of the CCL5 polypeptide. Orthologues of a CCL5 polypeptide are readily identified as comprising an amino acid sequence that is substantially homologous to SEQ ID NO:1. Programs such as BLAST (Basic Local Alignment Search Tool; Altschul et al., 1990), MCompIdent and MCompSim (Wilkens et al., 1998) are publicly available on the ExPASy (Expert Protein Analysis System) proteomics server of the Swiss Institute of Bioinformatics (SIB) and are particularly useful for identifying homologous polypeptides in public databases such as GenBank, Protein Data Bank (PDB), SwissProt, Protein Information Resource (PIR) and Protein Research Foundation (PRF).

As previously stated, chemokines (also known as chemotactic cytokines) are small molecular weight (e.g., about 8-10 kDa) polypeptides that play a central role in directing or recruiting the movement of cells (e.g., monocytes) towards a site of injury or infection. In certain embodiments, a CCL5 polypeptide (or a fragment thereof) is a chemically modified CCL5 polypeptide or a genetically modified CCL5 polypeptide, such that the modified CCL5 polypeptide is biologically inactive as a chemokine agonist in a mammalian subject. For example, a CCL5 modification is one which diminishes, reduces or inactivates the biological activity of the CCL5 polypeptide as a chemokine agonist, wherein the modified CCL5 polypeptide retains its ability to inhibit paramyxovirus infection. As defined herein, a “chemokine agonist” or the “biological activity of a chemokine or chemokine agonist” refers to the ability of a chemokine polypeptide (e.g., CCL5) to induce or stimulate chemotaxis, calcium mobilization, inflammation and the like.

The agonist activity of a chemokine polypeptide of the invention is detected or measured by methods such as calcium mobilization assays, chemotaxis assays, N-Acetyl-β-D-glucosaminidase assays and the like (Proudfoot et al., 1996; Simmons et al, 1997; Gong and Clark-Lewis, 1995; Fincham, 1988) (e.g., see Example 6).

In certain embodiments, a CCL5 polypeptide is genetically and/or chemically modified at its NH₂-terminus, wherein the NH₂-terminus modification inactivates CCL5 as a chemokine agonist. Several CCL5 NH₂-terminal modifications have been described in the art which inactivate CCL5 chemokine activity. For example, when the initiating methionine (Met) of CCL5 (RANTES) is retained, the resulting Met-CCL5 (Met-RANTES) polypeptide (1) is inactive as a chemokine agonist (e.g., Met-CCL5 does not stimulate or induce Ca²⁺ mobilization or chemotaxis), (2) antagonizes the “chemokine” effects induced by CCL5 and CCL3 (MIP-1α) at the CCR5 receptor (Proudfoot et al., 1996) and (3) inhibits HIV-1 infection of primary human macrophage cultures (Simmons et al, 1997). As defined hereinafter, a “Met-CCL5” or “Met-RANTES” polypeptide comprises an amino acid sequence of SEQ ID NO:1 and further comprises a methionine amino acid NH₂-terminal to the serine residue at position 1 of SEQ ID NO:1.

Truncated NH₂-terminus CCL5 polypeptides have also been described which inactivate CCL5 chemokine activity. For example, Arenzara-Seisdedos et al. (1996) synthesized a truncated CCL5 polypeptide in which the first eight amino acids were deleted (termed RANTES(9-68)), referred to hereinafter as “CCL5(Δ1-8)” and a truncated CCL5 polypeptide in which the first two amino acids were deleted (termed RANTES(3-68)), referred to hereinafter as “CCL5(Δ1-2)”, wherein the truncated polypeptides lacked chemotactic and leukocyte-activating properties and inhibited HIV-1 infection. As defined hereinafter, a “CCL5(Δ1-8)” polypeptide comprises amino acid residues 9 through 68 of SEQ ID NO:1. As defined hereinafter, a “CCL5(Δ1-2)” polypeptide comprises amino acid residues 3 through 68 of SEQ ID NO:1

Chemical modifications of CCL5 polypeptides also have been described which inactivate CCL5 chemokine activity. These modifications include the addition of an aminooxypentane group (referred to as “AOP-RANTES” or “AOP-CCL5”) to the NH₂-terminal serine residue of CCL5 (Simmons et al., 1997) and the addition of an N^{α}-nonanoyl group (referred to as “N^{α}-nonanoyl-RANTES”, “NNY-RANTES” or “NNY-CCL5”) to NH₂-terminal serine residue of CCL5 (Mosier et al., 1999). As defined hereinafter, an AOP-CCL5 polypeptide comprises an amino acid sequence of SEQ ID NO:1 and further comprises an aminooxypentane group covalently attached to the first serine residue of SEQ ID NO:1. As defined hereinafter, an NNY-CCL5 polypeptide comprises an amino acid sequence of SEQ ID NO:1 and further comprises an N^{α}-nonanoyl group covalently attached to the first serine residue of SEQ ID NO:1. Alternatively, the Ser1 residue is substituted with another amino acid (e.g., Gly1), wherein the substituted amino acid at position 1 comprises an AOP or NNY covalently attached.

Thus, in certain embodiments, modifications and changes are made in the primary sequence of a CCL5 polypeptide of the invention which inactivate CCL5 as a chemokine agonist, wherein the modified CCL5 retains its anti-paramyxovirus properties. For example, the functional and/or biological activity of a polypeptide is determined by complex interactions at the level of primary, secondary and tertiary structure, and as such, certain amino acid sequence substitutions may be made in a polypeptide sequence (or its underlying DNA coding sequence) and a polypeptide with similar properties (e.g., antiviral properties) is obtained.

In making such changes, the hydropathic index of amino acids are considered (e.g., see FIGS. 10A and 10B). The importance of the hydropathic amino acid index in conferring interactive biologic function on a polypeptide is generally understood in the art (e.g., Kyte and Doolittle, 1982; Eisenberg et al., 1984; Hopp and Woods, 1981). It is known that certain amino acids are substituted for other amino acids having a similar hydropathic index or score and still result in a polypeptide with similar biological activity. For example, the relative hydropathic character of an amino acid residue affects the secondary and tertiary structure of the resultant polypeptide, which in turn defines the interaction of the polypeptide with other molecules, such as enzymes, substrates, receptors, antibodies, antigens, and the like.

As outlined above, amino acid substitutions are generally based on the relative similarity of the amino acid side-chain substituents, for example, their hydrophobicity, hydrophilicity, charge, size, and the like. Exemplary substitutions which take various of the foregoing characteristics into consideration are well known to those of skill in the art and are set forth below in Table 1.

TABLE 1
AMINO ACID SUBSTITUTIONS

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

In certain embodiments, a CCL5 polypeptide is modified at its NH₂-terminus, wherein the NH₂-terminus modification inactivates CCL5 as a chemokine agonist. Thus, in certain of these embodiments, a CCL5 polypeptide is modified at its NH₂-terminus using site-specific mutagenesis (e.g., CCL5Δ1-8, Arenzana-Seisdedos et al., 1996). Alternatively, a CCL5 polypeptide (or a fragment thereof) is modified at its NH₂-terminus by chemical or synthetic modifications known in the art (e.g., AOP-CCL5, Simmons et al., 1997; NNY-CCL5, Mosier et al., 1999).

Site-specific mutagenesis is also useful in the preparation of second generation CCL5 polypeptides (e.g., an antiviral polypeptide (or fragment thereof) having reduced chemokine activity and/or a CCL5 polypeptide (or fragment thereof) having increased antiviral properties) through specific mutagenesis of the underlying DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered. The technique of site-specific mutagenesis is well known in the art and typically employs a phage vector which can exist in both a single stranded and double stranded form (e.g., see U.S. Pat. No. 5,556,747; U.S. Pat. No. 5,789,166 and U.S. Pat. No. 6,391,548, each incorporated by reference herein in its entirety).

In particular embodiments, the invention is directed to CCL5 polypeptide fragments. As used herein, the terms “polypeptide fragment”, “peptide fragment” or “protein fragment” are used interchangeably. As defined hereinafter, a CCL5 peptide fragment is a CCL5 polypeptide having an amino acid sequence that is entirely the same as part, but not all, of the full length or mature CCL5 amino acid sequence. Thus, a polypeptide fragment comprises, for example, at least seven or more (e.g., 8, 10, 12, 14, 16, 18, 20, or more) contiguous amino acids of a CCL5 polypeptide of SEQ ID NO:1. A CCL5 peptide fragment is produced or generated via recombinant expression methods, synthetic peptide chemistry or by chemical cleavage or enzymatic cleavage of a full length CCL5 polypeptide. Fragments are “freestanding” or comprised within a larger polypeptide of which they form a part or region, most preferably as a single, continuous region. In one embodiment, a CCL5 peptide fragment comprises about ten to twenty contiguous amino acids (e.g., a 10-mer, an 11-mer, a 12-mer, a 13-mer, a 14-mer, a 15-mer, etc.) of the full length CCL5 polypeptide of SEQ ID NO:1.

As described in Example 4, a series of nine overlapping CCL5 peptide fragments (15-mers) were generated (see, Table 2 below) and tested via in vitro and in vivo RSV infection assays. It was observed in these assays, that peptide number 1 (SEQ ID NO:2), representing the first fifteen NH₂-terminal amino acids of CCL5 (i.e., amino acids 1-15 of SEQ ID NO:1), was the most inhibitory of the nine overlapping CCL5 peptides tested. It is also known in the art of chemokines, that NH₂-terminal CCL5 peptide fragments inhibit HIV-1 infection of lymphocytes. For example, Nardese et al. (2001) designed a series of synthetic 12-mer peptides encompassing both the NH₂-loop and β1-strand residues that form the hydrophobic patch on CCL5 (e.g., see Section A.2 and Table 5A below), a 19-mer spanning both the NH₂-loop and P1-strand aromatic cluster and a cyclic 24-mer spanning Cys11-Cys43. These peptides were acylated (Ac) and amidated (NH₂) at the amino acid position indicated below. In an acute infection assay, the anti-HIV-1 potency of the 19-mer spanning both the NH₂-loop and P1-strand aromatic cluster (Ac-Cys11-Tyr29-NH₂; SEQ ID NO:16) was significantly higher than that of an NH₂-loop-derived dodecamer Ac-Cys11-Ala22-NH₂ (SEQ ID NO:13) (mean ID₅₀=8.9+/−3.8 μM versus 51.5+/−6.3 μM; P<0.0001). Two shorter peptides lacking the β1-strand aromatic cluster (Cys11-Lys25 and Cys11-Glu26) were less effective (mean ID₅₀=48.1+/−6.8 μM and 44.7+/−3.3 μM, respectively), confirming the importance of such an aromatic cluster for the HIV-1 antiviral activity. A cyclic 24-mer spanning the entire region between the second and third cysteines (cylic-Cys1′-Cys34, SEQ ID NO:17) inhibited HIV-1 more potently than the original dodecamer (SEQ ID NO:13) (mean ID₅₀=29.8+/−1.6 μM), although less than the 19-mer (SEQ ID NO:16). No effect was seen with a co-linear control cyclic peptide derived from the CXCR4 ligand SDF-1. These findings demonstrated that the aromatic cluster within the β1 strand of CCL5 is crucial for the anti-HIV-1 activity, confirming that this region, along with the NH₂-loop, is involved in the CCR5 receptor interface.

The CCL5 peptide fragments which inhibited RSV infection (Table 2; peptide number 1) and HIV-1 infection (Table 3) were aligned and are shown in Table 4 below. Also presented in Table 4 (bold text) is a consensus CCL5 peptide fragment (peptide No. 17) comprising an amino acid sequence of SEQ ID NO:18.

TABLE 2

AMINO ACID SEQUENCES OF OVERLAPPING CCL5
SYNTHETIC PEPTIDE PENTADECAMERS (15-MERs)

| | | | |
|---|---|---|---|
| | | | SEQ |
| Peptide | Amino | | ID |
| Number | Acids | Sequence | NO |

| | | | |
|---|---|---|---|
| 1 | 1-15 | SPYSSDTTPCCFAYI | 2 |
| 2 | 8-22 | TPCCFAYIARPLPRA | 3 |
| 3 | 15-29 | IARPLPRAHIKEYFY | 4 |
| 4 | 22-36 | AHIKEYFYTSGKCSN | 5 |
| 5 | 29-43 | YTSGKCSNPAVVFVT | 6 |
| 6 | 37-50 | PAVVFVTRKNRQVC | 7 |
| 7 | 43-57 | TRKNRQVCANPEKKW | 8 |
| 8 | 50-64 | CANPEKKWVREYINS | 9 |
| 9 | 54-68 | EKKWVREYINSLEMS | 10 |

TABLE 3

AMINO ACID SEQUENCES OF ANTI-HIV CCL5
DODECAMERS (12-MERS), A CCL5 NONADECAMER
(19-MER) AND A CYCLIC 24-MER

| | | | SEQ |
|---|---|---|---|
| Peptide | Amino | | ID |
| Number | Acids | Sequence | NO |
| 10 | 7-18 | TTPCCFAYIARP | 11 |
| 11 | 9-20 | PCCFAYIARPLP | 12 |
| 12 | 11-22 | CFAYIARPLPRA | 13 |
| 13 | 23-34 | HIKEYFYTSGKC | 14 |
| 14 | 31-42 | SGKCSNPAVVFV | 15 |
| 15 | 11-29 | CFAYIARPLPRAHIKEYFY | 16 |
| 16 | 11-34 | CFAYIARPLPRAHIKEYFYTSGKC | 17 |

TABLE 4

AMINO ACID SEQUENCE ALIGNMENT OF ANTI-RSV
AND ANTI-HIV-1 CCL5 FRAGMENTS AND THE
CONSENSUS AMINO ACID SEQUENCE

| | | | |
|---|---|---|---|
| Pep- | | | SEQ |
| tide | Amino | Alignment of Amino | ID |
| Number | Acids | Acid Sequences | NO |

| | | | |
|---|---|---|---|
| 1 | 1-15 | SPYSSDTTPCCFAYI | 2 |
| 10 | 7-18 | TTPCCFAYIARP | 11 |
| 11 | 9-20 | PCCFAYIARPLP | 12 |
| 12 | 11-22 | CFAYIARPLPRA | 13 |
| 13 | 23-34 | HIKEYFYTSGKC | 14 |
| 15 | 11-29 | CFAYIARPLPRAHIKEYFY | 16 |
| 16 | 11-34 | CFAYIARPLPRAHIKEYFYTSGKC | 17 |
| 17 | 1-34 | SPYSSDTTPCCFAYIARPLPRAHIKEYFYTSGKC | 18 |

Thus, in certain embodiments, a CCL5 peptide fragment comprises an amino acid sequence of SEQ ID NO:2, representing the NH₂-terminal fifteen residues of a full length CCL5 polypeptide. In other embodiments, the CCL5 peptide of SEQ ID NO:2 is modified at one or more of its NH2-terminal amino acid residues. In other embodiments, a CCL5 peptide fragment comprises an amino acid sequence of SEQ ID NO:18 or an NH₂-terminally modified fragment thereof. In certain other embodiments, the primary and secondary sequence of a CCL5 peptide fragment of SEQ ID NO:2, SEQ ID NO:11, SEQ ID NO:12, SEQ ID NO:13, SEQ ID NO:14, SEQ ID NO:15, SEQ ID NO:16, SEQ ID NO:17 or SEQ ID NO:18, is used to design a peptide mimetic or a organic small molecule mimetic as set forth in below in Section A.2.

It is contemplated in certain embodiments of the invention, that a CCL5 polypeptide is cleaved into fragments by chemical or enzymatic cleavage. This is accomplished by treating purified CCL5 polypeptides with a proteolytic enzyme (i.e., a proteinase) including, but not limited to, serine proteinases (e.g., chymotrypsin, trypsin, plasmin, elastase, thrombin, substilin) metal proteinases (e.g., carboxypeptidase A, carboxypeptidase B, leucine aminopeptidase, thermolysin, collagenase), thiol proteinases (e.g., papain, bromelain, Streptococcal proteinase, clostripain) and/or acid proteinases (e.g., pepsin, gastricsin, trypsinogen). Polypeptide fragments also are generated using chemical means such as treatment of the polypeptide with cyanogen bromide (CNBr), 2-nitro-5-thiocyanobenzoic acid, isobenzoic acid, BNPA-skatole, hydroxylamine or a dilute acid solution. In other embodiments, the CCL5 polypeptide fragments of the invention are recombinantly expressed or prepared via peptide synthesis methods known in the art (Barany et al., 1997; Simmons et al., 1997; Proudfoot et al., 1996; Proudfoot et al., 1999; U.S. Pat. No. 5,258,454) and described in Example 1.

2. Peptide Mimetics and Organic Small Molecule Mimetics

A common approach to drug design involves the examination of protein-protein interactions associated with a particular disease, followed by the design of small molecules that can mimic or bind to one of the interacting proteins. Often the bioactivity stems from only a small localized region of a protein surface created by secondary structural elements such as α-helices, β-sheets, β-turns, γ-turns, β-strands, loop structures and the like. Thus, small organic molecule mimetics and peptide mimetics are often designed to exert their biological activity by mimicking these localized structural elements (i.e., secondary structure) of the proteins folded surface (i.e., tertiary structure).

A “peptide mimetic” or “peptidomimetic” refers to various types or classes of molecules, as long as the resulting molecule mimics or resembles a desired polypeptide secondary (or localized tertiary) structural element. For example, a peptide mimetic is an oligomer that mimics peptide secondary structure through use of amide bond isosteres and/or modification of the native peptide backbone, including chain extension or heteroatom incorporation; examples of which include azapeptides, oligocarbamates, oligoureas, β-peptides, γ-peptides, oligo(phenylene ethynylene)s, vinylogous sulfonopeptides, poly-N-substituted glycines (peptoids) and the like (e.g., see Gellman, 1998; Kirshenbaum et al., 1999; Barron and Zuckermann, 1999). Methods for designing and synthesizing peptide mimetics are well known to one of skill in the art. In certain embodiments, it is contemplated that a peptide mimetic is used to overcome protease sensitivity, stabilize secondary structure and/or improve bioavailability relative to a naturally occurring CCL5 peptide analogues. In certain embodiments, a peptide mimetic of the invention is a reverse turn mimetic, e.g., a α-turn mimetic, a monocyclic β-turn mimetic, a bicyclic β-turn mimetic, a γ-turn mimetic or a monocyclic γ-turn mimetic.

β-strand secondary structure has often been considered a random conformation, but more recently it has been recognized as a fundamental and discrete element of protein structure recognized by a wide range of biomolecular receptors. For example, it has been convincingly demonstrated that all proteolytic enzymes bind their inhibitors/substrates in extended β-strand structures, in which the peptide backbone or equivalent non-peptide molecule is in a linear confirmational arrangement (Tyndall and Fairlie, 1999; Fairlie et al., 2000; Bode and Huber, 1992). A β-turn is a site in a polypeptide structure where the polypeptide chain reverses its direction. These turns are polar and located primarily on the surfaces of the protein molecule (i.e., solvent exposed) and thus serve as ideal sites for receptor binding, antibody recognition and peptide mimetic design.

Thus, in certain embodiments, a molecule which inhibits or prevents paramyxovirus infection in a mammalian subject is a peptide mimetic or an organic small molecule mimetic (hereinafter, a “small molecule” or “small molecule mimetic”). A peptide mimetic or small molecule mimetic of the invention is designed to mimic or resemble certain secondary or tertiary structural elements of a CCL5 polypeptide or a modified CCL5 polypeptide thereof (e.g., Met-CCL5; AOP-CCL5) as described below.

The three-dimensional solution structure of CCL5 has been solved via two- and three-dimensional NMR (Skelton et al., 1995). The NMR data obtained was used to generate a twenty member ensemble of energy minimized CCL5 structures, which were deposited in the Brookhaven Protein Data Bank under the accession name 1RTN. A mean CCL5 structure, calculated by 2000 steps of in silico energy minimization using the twenty CCL5 structures (Skelton et al., 1995), was also deposited (PDB accession name 1RTO). More recently, AOP-CCL5 has been chemically synthesized and its high resolution (1.6 Å) crystal structure solved and deposited under PDB accession name 1B3A (Wilken et al., 1999), followed shortly thereafter by the high resolution (1.6 Å) crystal structure of Met-CCL5, deposited under PDB accession name 1 EQT (Hoover et al., 2000).

The polypeptide fold of CCL5 is similar to that of other CC and CXC polypeptides, forming a three-stranded antiparallel β-sheet flanked by a COOH-terminal α-helix. Listed in Table 5A and Table 5B below are the secondary structural elements of CCL5 and AOP-CCL5, as determined by NMR (PDB 1RTO; Skelton et al., 1995) and X-ray crystallography (PDB 1 B3A; Wilken et al., 1999), respectively.

TABLE 5A
CCL5 SECONDARY STRUCTURE

| CCL5 | |
|---|---|
| Amino Acids | 2° Structure |
| Ser1-Ser4 | Extended, disordered |
| Ser5-Cys10 | β-sheet (β0) |
| Cys11-His23 | NH₂-loop |
| Ile24-Ser31 | β-sheet (β1) |
| Gly32-Ala38 | loop |
| Val39-Thr43 | β-sheet (β2) |
| Arg44-Asn46 | loop |
| Arg47-Ala51 | β-sheet (β3) |
| Asn52-Lys55 | loop |
| Lys56-Glu66 | α-helix |
| Met67-Ser68 | extended |

TABLE 5B
AOP-CCL5 Secondary Structure

| AOP-CCL5 | |
|---|---|
| Amino Acids | 2° Structure |
| Ser1-Thr7 | β-strand or extended |
| Thr8-Cys10 | β-sheet (β0) |
| Cys11-Pro20 | loop |
| Arg21-His23 | α helix |
| Ile24-Tyr29 | β-sheet (β1) |
| Thr30-Ala39 | loop |
| (Ser31-Cys34) | (type III reverse turn) |
| Val39-Thr43 | β-sheet (β2) |
| Thr43-Arg47 | loop |
| (Thr43-Asn46) | (type I reverse turn) |
| Gln48-Ala51 | β-sheet (β3) |
| Asn52-Lys55 | type I reverse turn |
| Lys56-Glu66 | α helix |
| Met67-Ser68 | extended |

The NMR solution structure of the CCL5 dimer (Skelton et al., 1995) shows a partially disordered NH₂-terminal region followed by a short-strand (β0) leading to the signature two-cysteine motif, an extended region (NH₂-loop) ending with a 3₁₀ turn, three antiparallel β-strands (β1-β3) connected by loops and a COOH-terminal α-helix. The physiological relevance of chemokine dimers is still debated, but recent studies indicate that the biological function of CCL5 is dependent on the dimeric structure (Nardese et al., 2001). According to the two-site chemokine model, two distinct regions of the chemokines participate in the interaction with chemokine receptors: the NH₂-terminus, which is critical for receptor activation, and another domain responsible for the primary docking event, which was suggested to involve the NH₂-loop region (Clark-Lewis, 1994; Schraufstätter, 1995; Lowman et al., 1996; Pakianathan et al., 1997; Crump et al., 1997; Hemmerich et al., 1999; Laurence et al., 2000). The NH₂-loop is believed to play a pivotal role in the physiology of HIV-1 infection, because neither chemokine-mediated HIV-1 blockade nor the HIV-1 co-receptor function requires the signaling activity of chemokine receptors (Nardese et al., 2001).

The functional mapping of CCL5 (described above in Section A.1) was correlated with the NMR solution structure (Nardese et al., 2001). Analysis of the surface electrostatic potential of the CCL5 dimer indicated that the NH₂-loop residues critical for the HIV-1 antiviral activity (Phe12, Tyr14 and Ile15) are displayed on the surface of the molecule, where they contribute to the formation of a large (180 Å²) hydrophobic, solvent-exposed patch. This structural feature is consistent with a potential role as a receptor-docking site. For example, Phe12 is structurally equivalent in the related CC chemokine MCP-1 to Tyr13, a residue implicated in CCR2 binding and contributing, along with other NH₂-loop residues, to the formation of a hydrophobic surface groove. Similarly, the equivalent residue in MIP-1P, Phe13, is critical for CCR5 binding and chemokine dimerization. However, the hydrophobic patch on CCL5 also encompasses a cluster of aromatic residues (Tyr27-Tyr29) lying at the center of the β1-strand peptide that displayed HIV-1 antiviral activity (e.g., see Section A.1 above). Thus, the structural analysis of Nardese et al. suggests that both the NH₂-loop and P1-strand residues contribute to the formation of the putative CCR5 receptor interface. Interestingly, the P1-strand residues of CCL5 are not required for the inhibition of RSV infection of mammalian epithelial cells, suggesting the possibility of an alternate mechanism (or alternate structural elements) for CCL5 inhibition of RSV infection (i.e., relative to HIV-1 infection).

Peptide mimetics with inverted amino acid chirality (i.e., D-amino acid stereoisomers of the naturally occurring L-amino acid stereoisomers) are inherently resistant to protease-mediated digestion and thus represent suitable candidates for therapeutic applications. Nardese et al. (2001) synthesized an inverted mimetic of an NH₂-loop/β1-strand 19-mer with a reversed direction of the peptide bonds in order to preserve the original side chain topochemistry. The retroinverted peptide (Ac-D-Tyr29-D-Cys11-NH₂; SEQ ID NO:19) effectively inhibited HIV-1 envelope-mediated cell fusion with a potency (mean ID₅₀=13.3+/−2.7 μM) comparable to that of its L-amino acid counterpart (SEQ ID NO:16) and it antagonized chemotaxis. By contrast, no effect was seen with a control retroinverted peptide with similar amino acid composition and hydrophobicity value. These biological activities confirmed the prediction that retroinversion resulted in the preservation of the original spatial orientation of the peptide lateral side chains.

Thus, in certain embodiments, the invention is directed to a peptide mimetic of a CCL5 polypeptide from about amino acid residue one to about amino acid residue thirty of SEQ ID NO:1, wherein the peptide mimetic inhibits paramyxovirus infection in a mammalian subject. In one particular embodiment, a peptide mimetic is based on amino acid residues one through fifteen of the NH₂-terminal CCL5 peptide fragment of SEQ ID NO:1, as represented by SEQ ID NO:2 and shown to inhibit RSV infection (Example 4). In another embodiment, a peptide mimetic is based on amino acid residues one through twenty-two of SEQ ID NO:1. In another embodiment, a peptide mimetic is based on amino acid residues one through twenty-nine of SEQ ID NO:1. In another embodiment, a peptide mimetic is a retroinverted peptide comprising amino acid residues eleven through twenty-nine of SEQ ID NO:1 in reverse order as follows: Ac-YFYEKIHARPLPRAIYAFC-NH₂ (SEQ ID NO:19), represented hereinafter as “Ac-D-Tyr29-D-Cys11-NH₂”. In another embodiment, a peptide mimetic is a retroinverted peptide comprising amino acid residues one through thirty-four of SEQ ID NO:1 in reverse order as follows: Ac-CKGSTYFYEKIHARPLRPAIYAFCCPTTDSSYPS-NH₂ (SEQ ID NO:20), represented hereinafter as “Ac-D-Cys34-D-Ser1-NH₂”. In still another embodiment, a peptide mimetic is a retroinverted peptide comprising amino acid residues one through fifteen of SEQ ID NO:1 in reverse order as follows: Ac-IYAFCCPTTDSSYPS-NH₂ (SEQ ID NO:21), represented hereinafter as “Ac-D-Iso15-D-Ser1-NH₂”. In another embodiment, a peptide mimetic or a small molecule mimetic comprises the secondary and tertiary structural elements of the NH₂-loop amino acids Phe12, Tyr14 and Ile15. These structural elements are readily determined via in silico molecular modeling and visualization of a Brookhaven Protein Data Bank molecular coordinate accession file selected from the group consisting of 1EQT (Met-CCL5), 1B3A (AOP-CCL5) and 1RTO (CCL5), as described below. In still other embodiments, the molecular coordinates of CCL5 (PDB 1RTN; PDB 1RTO), Met-CCL5 (PDB 1EQT) and/or AOP-CCL5 (PDB 1B3A) are used to generate (via in silico molecular modeling) second generation peptide mimetics of SEQ ID NO:1, SEQ ID NO:2, SEQ ID NO:18 or an NH₂-terminal fragment thereof.

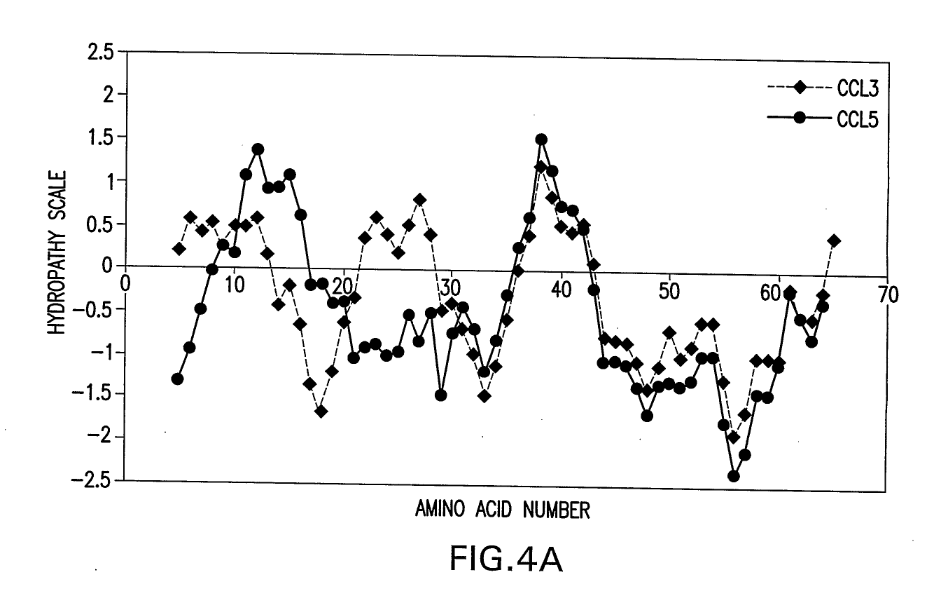

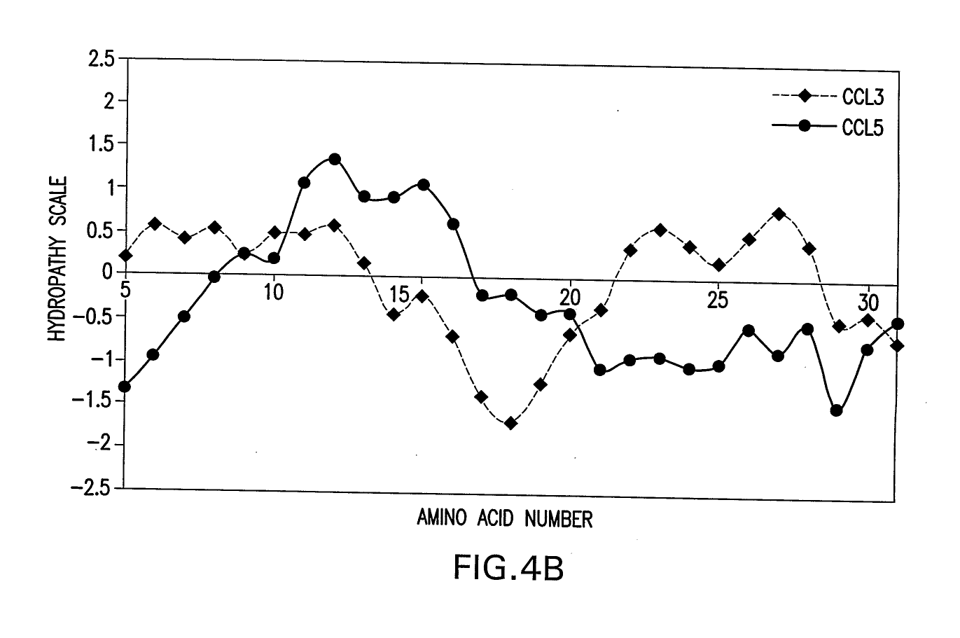

As stated previously, the polypeptide fold of CCL5 is similar to that of other CC and CXC chemokines (e.g., CCL3, CCL4). However, it was demonstrated in the present invention that CCL5, but not CCL3 (MIP-1a) or CCL4 (MIP-1β), inhibited RSV infection of epithelial cells, suggesting that RSV (in contrast to HIV-1) may utilize a receptor other than CCR5. To further elucidate the sequence and/or structural requirements of CCL5 mediated inhibition of RSV infection, CCL5 and CCL3 polypeptides were compared by amino acid sequence alignment (Table 6), hydropathy plots (FIG. 4A and FIG. 4B) and molecular modeling/visualization (FIG. 11). As described in Example 5, a comparison of CCL5 and CCL3 (MIP-1α) sequences indicated that CCL5 and CCL3 share 32 identical amino acids (i.e., 48% identity) and have about 78% amino acid sequence similarity (Table 6).

TABLE 6
BLAST ALIGNMENT OF CCL5 AND CCL3

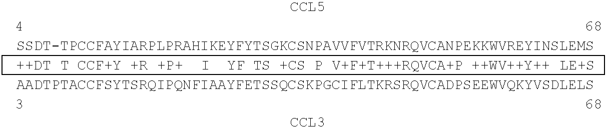

A comparison of the hydropathy plots (Kyte and Doolittle, 1982) of full length CCL5 versus full length CCL3 (FIG. 4A and FIG. 4B) indicated that the greatest hydropathic sequence divergence between CCL5 and CCL3 occurs within NH₂-terminus of these polypeptides (e.g., see Example 5). The energy minimized structures of CCL5 (PBD 1RTO) and CCL3 (PBD 1B53) were modeled in silico (SWISS-MODEL and Swiss-PdbViewer (Guex and Peitsch, 1997)) and the tertiary structures (or fold) superimposed with the exception of the NH₂-terminal amino acids 1 to 7 (data not shown) and COOH-terminal amino acids 64 to 68. Similar to the CCL5/CCL3 hydropathy plots (FIG. 4A), the greatest structural sequence divergence between CCL5 and CCL3 occurs in the NH₂-terminal amino acid residues. Without wishing to be bound by any particular theory, it is contemplated that the difference between CCL5 inhibition of HIV-1 infection relative to the inhibition of RSV, may in part be modulated by the first fifteen amino acids of the CCL5 NH₂-terminus.

Thus, in certain embodiments, a peptide mimetic or a small molecule mimetic of the invention mimics a CCL5 NH₂-terminal peptide fragment comprising an amino acid sequence of SEQ ID NO:2. In one particular embodiment, a peptide mimetic is based on the dihedral angles of the first fifteen amino acids of CCL5, AOP-CCL5 or Met-CCL5 as set forth in Table 7.

TABLE 7
THE DIHEDRAL ANGLES FOR THE FIRST FIFTEEN NH2-TERMINAL
AMINO ACIDS OF CCL5, MET-CCL5 AND AOP-CCL5

| | Phi | Psi | AOP- | Phi | Psi | Met- | Phi | Psi |
|---|---|---|---|---|---|---|---|---|
| CCL5 | Angle | Angle | CCL5 | Angle | Angle | CCL5 | Angle | Angle |
| Ser1 | — | — | AOP1 | — | — | Met1 | — | — |
| Pro2 | −78 | −89 | Pro2 | — | — | Gly2 | — | — |
| Tyr3 | −162 | +64 | Tyr3 | −119 | −137 | Tyr3 | −122 | −171 |
| Ser4 | −87 | +60 | Ser4 | −64 | +135 | Ser4 | −59 | +146 |
| Ser5 | −90 | +52 | Ser5 | −101 | +10 | Ser5 | −101 | +11 |
| Asp6 | −139 | +165 | Asp6 | −78 | +153 | Asp6 | −83 | +156 |
| Thr7 | −72 | +160 | Thr7 | −68 | +158 | Thr7 | −69 | +161 |
| Thr8 | −114 | +132 | Thr8 | −139 | +145 | Thr8 | −141 | +142 |
| Pro9 | −78 | +91 | Pro9 | −72 | +133 | Pro9 | −75 | +135 |
| Cys10 | −106 | +117 | Cys10 | −122 | +158 | Cys10 | −122 | +161 |
| Cys11 | −103 | +129 | Cys11 | −109 | +137 | Cys11 | −112 | +142 |
| Phe12 | −118 | −19 | Phe12 | −121 | −5 | Phe12 | −126 | −2 |
| Ala13 | −90 | +165 | Ala13 | −163 | +158 | Ala13 | −156 | +153 |
| Tyr14 | −140 | +104 | Tyr14 | −118 | +137 | Tyn14 | −110 | +141 |
| Iso15 | −60 | +125 | Iso15 | −64 | +139 | Iso15 | −62 | +136 |

In other embodiments of the invention, an antiviral molecule is a non-peptide small molecule mimetic (in contrast to a peptide mimetic) which inhibits or prevents paramyxovirus infection of a mammalian cell. Similar to the design of a peptide mimetic, a non-peptide small molecule of the invention is designed to mimic the key structural elements or amino acid residues of the CCL5 polypeptide of SEQ ID NO:1. In one particular embodiment, a small molecule is based on amino acid residues one through fifteen of the NH₂-terminal CCL5 peptide fragment of SEQ ID NO:1 as represented by SEQ ID NO:2. In another embodiment, a small molecule is based on amino acid residues eleven through twenty-two of SEQ ID NO:1 as represented by SEQ ID NO:13. In another embodiment, a small molecule is based on amino acid residues eleven through twenty-nine of SEQ ID NO:1 as represented by SEQ ID NO:16. In another embodiment, a small molecule is based on the retroinverted peptide mimetic Ac-D-Tyr29-D-Cys11-NH₂ of SEQ ID NO:18.

In a preferred embodiment, a small molecule of the invention mimics the CCL5 hydrophobic patch formed by amino acid residues Phe12, Ala13, Tyr14 and Ile15 of the NH2-loop. In certain embodiments, the small molecule mimetic comprises the three-dimensional molecular arrangement of amino acids Phe12, Tyr14 and Ile15 as found in a functional and folded CCL5 polypeptide, wherein Phe12, Tyr14 and Ile15 are spatially constrained according to the dihedral angles of the molecular coordinates of CCL5 (PDB 1RTO), the molecular coordinates of AOP-CCL5 (PDB 1B3A) or Met-CCL5 (PDB 1EQT). In other embodiments, a small molecule of the invention mimics the CCL5 hydrophobic patch formed by amino acid residues Phe12, Ala13, Tyr14 and Ile15 of the NH2-loop and residues Tyr27, Phe28 and Tyr29 of the P1-sheet, as described in Nardese et al. (2001). In certain other embodiments, the molecular coordinates of CCL5 (PDB 1RTN; PDB 1TRO), Met-CCL5 (PDB 1EQT) and/or AOP-CCL5 (PDB 1B3A) are used to generate (via in silico molecular modeling and computations) a small molecule which mimics the CCL5 hydrophobic patch.

As stated above, certain data of the invention indicate that CCR3 is a co-receptor for RSV entry into mammalian epithelial cells. Thus, in another embodiment the invention is directed to an antiviral molecule of the invention administered in combination with a small molecule antagonist of the CCR3 receptor, a peptide antagonist of the CCR3 receptor, a mimetic of the CCR3 receptor or a combination thereof. In certain embodiments, a CCR3 antagonist is a molecule comprising one or more of the following chemical structures:

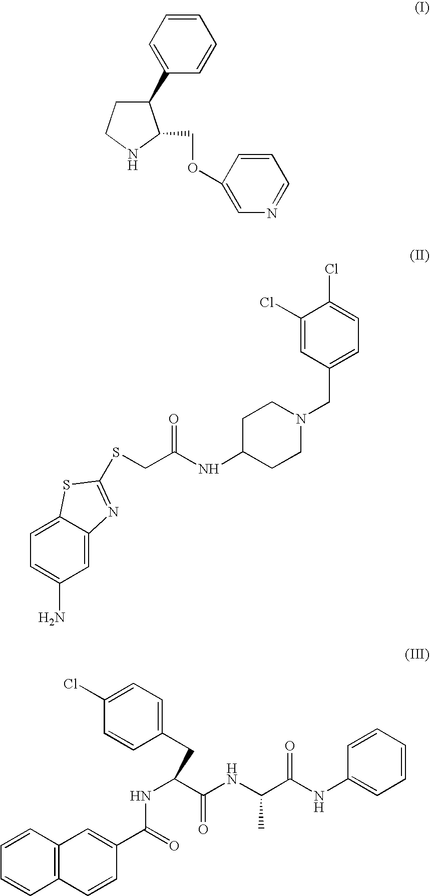

In other embodiments, an antiviral molecule of the invention is administered in combination with a small molecule antagonist of the CCR1 receptor. In certain embodiments, a CCR1 antagonist is molecule comprising one or more of the following chemical structures:

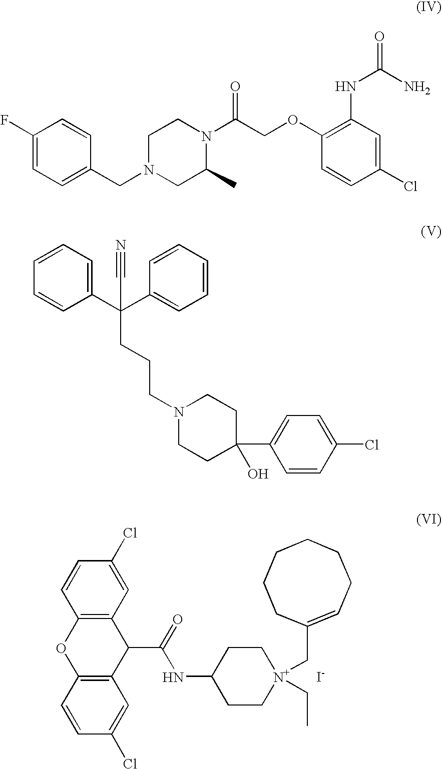

In still other embodiments, an antiviral molecule of the invention is administered in combination with a small molecule antagonist of the CCR5 receptor. In certain embodiments, a CCR5 antagonist is molecule comprising one or more of the following chemical structures: 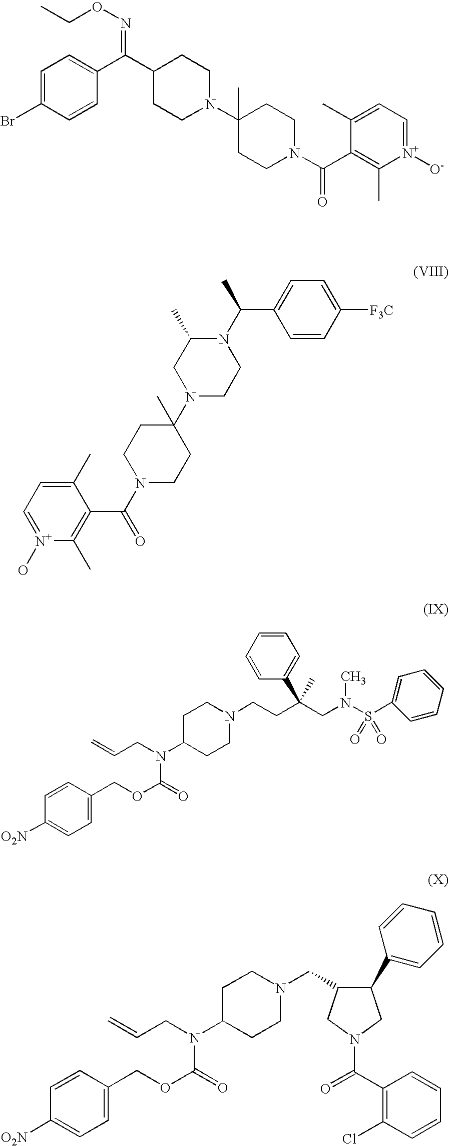 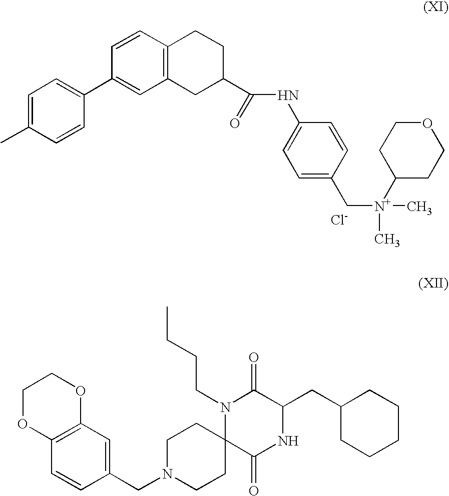
B. POLYNUCLEOTIDES ENCODING CHEMOKINES, RECOMBINANT EXPRESSION VECTORS AND HOST CELLS

In certain embodiments, a chemokine polypeptide (e.g., CCL5, CCL3), a truncated chemokine polypeptide (e.g., CCL5Δ1-8), a chemokine fragment (e.g., CCL5 of SEQ ID NO:2) and the like is recombinantly expressed. In a preferred embodiment, a polynucleotide encoding a chemokine polypeptide is comprised in a plasmid vector and expressed in a prokaryotic host cell. A polynucleotide sequence encoding a full length CCL5 polypeptide of the invention is set forth as SEQ ID NO:23.

When chemokine polynucleotides are used for the recombinant production of chemokine polypeptides, fragments thereof or truncations thereof, the polynucleotide includes the coding sequence for the polypeptide, or the coding sequence for the polypeptide in reading frame with other coding sequences, such as those encoding a leader or secretory sequence, a pre-, a pro- a prepro-protein sequence, or other fusion peptide portions. For example, a marker sequence which facilitates purification of the fused polypeptide can be linked to the coding sequence (see Gentz et al., 1989, incorporated by reference hereinafter in its entirety). Thus, contemplated in the present invention is the preparation of polynucleotides encoding fusion polypeptides permitting His-tag purification of expression products. The polynucleotide may also contain non-coding 5′ and 3′ sequences, such as transcribed, non-translated sequences, splicing and polyadenylation signals.

As used herein, the term “vector” refers to a nucleic acid molecule capable of transporting another nucleic acid to which it has been linked. A vector of the invention includes vectors known in the art as plasmids, viral vectors and the like. Expression of proteins in prokaryotes is most often carried out in E. coli with vectors containing constitutive or inducible promoters directing the expression of either fusion or non-fusion proteins. Fusion vectors add a number of amino acids to a protein encoded therein, to the amino or carboxy terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson, 1988), pMAL (New England Biolabs, Beverly; MA) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., 1988) and pET lid (Studier et al., 1990).

In certain embodiments, a recombinant expression vector is introduced into a “host cell” wherein the chemokine polypeptide is expressed. A “genetically engineered host cell” and “recombinant host cell” are used interchangeably herein. A host cell can be any prokaryotic or eukaryotic cell. For example, a chemokine polypeptide is expressed in bacterial cells such as E. coli, Moraxella catarrhalis, insect cells (such as Sf9 or Sf21 cells), yeast (such as S. cerevisiae) or mammalian cells (such as Chinese hamster ovary cells (CHO), NIH3T3, PER.C6, NSO or COS cells). Other suitable host cells are known to those skilled in the art.

Vector DNA is introduced into prokaryotic or eukaryotic cells via conventional transformation, infection or transfection techniques. As used herein, the terms “transformation”, “infection”, and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, infection or electroporation. Suitable methods for transforming, infecting or transfecting host cells can be found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, is also used to produce (i.e., express) large quantities of a desired chemokine polypeptide. Accordingly, the invention further provides methods for producing chemokine polypeptides using the host cells of the invention. In one embodiment, the method comprises culturing the host cell of invention (into which a recombinant expression vector encoding a polypeptide has been introduced) in a suitable medium until the chemokine polypeptide is produced. In another embodiment, the method further comprises isolating the chemokine polypeptide from the medium or the host cell.

An expression vector of the present invention is useful both as a means for preparing quantities of DNA encoding a chemokine polypeptide itself, and as a means for preparing the encoded polypeptides. It is contemplated that where chemokine polypeptides of the invention are made by recombinant means, one can employ either prokaryotic or eukaryotic expression vectors as shuttle systems.

C. ANTIVIRAL PHARMACEUTICAL COMPOSITIONS

In certain preferred embodiments, the present invention provides pharmaceutical compositions comprising an antiviral molecule of the invention and a pharmaceutically acceptable carrier. The antiviral molecules are incorporated into pharmaceutical compositions suitable for administration to a mammalian subject, e.g., a human. Such compositions typically comprise the “active” composition (i.e., the antiviral molecule) and a “pharmaceutically acceptable carrier”. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral (e.g., intravenous, intradermal, subcutaneous, intramuscular, intraperitoneal), mucosal (e.g., oral, rectal, intranasal, buccal, vaginal, respiratory) and transdermal (topical). Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid and the like. In many cases, it is preferable to include isotonic agents, for example, sugars, polyalcohols such as manitol, sorbitol, sodium chloride in the composition. Prolonged absorption of the injectable compositions is brought about by including in the composition an agent which delays absorption, for example, aluminum monostearate and gelatin.

Sterile injectable solutions are prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound is incorporated with excipients and used in the form of tablets, troches, or capsules. Oral compositions are also prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

The compounds are also prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials are also obtained commercially from Alza Corporation and Nova Pharmaceuticals, Inc. Liposomal suspensions (including liposomes targeted to infected cells with monoclonal antibodies to viral antigens) are also used as pharmaceutically acceptable carriers. These are prepared according to methods known to those skilled in the art, for example, as described in U.S. Pat. No. 4,522,811 which is incorporated hereinafter by reference.

It is especially advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

A pharmaceutically acceptable vehicle is understood to designate a compound or a combination of compounds entering into a pharmaceutical composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

A composition of the present invention is typically administered parenterally in dosage unit formulations containing standard, well-known nontoxic physiologically acceptable carriers, adjuvants, and vehicles as desired. The term parenteral as used hereinafter includes intravenous, subcutaneous, intradermal, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Preferred carriers include neutral saline solutions buffered with phosphate, lactate, Tris, and the like. When administering viral vectors, the vector is purified sufficiently to render it essentially free of undesirable contaminants, such as defective interfering adenovirus particles or endotoxins and other pyrogens, so that it does not cause any untoward reactions in the individual receiving the vector construct. A preferred means of purifying the vector involves the use of buoyant density gradients, such as cesium chloride gradient centrifugation.

A carrier can also be a liposome. Means for using liposomes as delivery vehicles are well known in the art.

All patents and publications cited herein are hereby incorporated by reference.

### D. EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Materials and Methods

Chemokines and reagents. Unless indicated otherwise, all recombinant (r) human chemokines and anti-human chemokine receptor antibodies were purchased from R & D Systems Inc. (Minneapolis, Minn.). Recombinant human CCL5 and stromal cell derived factor (SDF)-1α (CXCL12) were obtained respectively from Biosource International (Camarillo, Calif.) and Wyeth (Andover, Mass.). Both monoclonal and polyclonal antibodies against CC chemokine receptors (CCR) were used in the studies. The monoclonal antibodies (mAb) were directed against human CCR3 (catalog No. MAB155) or CCR5 (catalog No. MAB182). Affinity purified polyclonal antibodies raised against peptides sequences from CCR1 (catalogue No. sc-7934), CCR3 (catalog No. sc-7897), CCR4 (catalog No. sc-6126) or CCR5 (catalog No. sc-8283) were purchased from Santa Cruz Biotechnology (Santa Cruz, Calif.). Anti human CX3CR1 polyclonal antibody (catalogue No. TP502AF) was purchased from Torrey Pines Biolabs (Houston, Tex.). A series of nine overlapping synthetic peptides (Table 1) of CCL5 (Schall et al. 1988) were purchased from Sigma-Genosys (The Woodlands, Tex.) or synthesized using standard peptide chemistries. The peptides were labeled with biotin using EZ-LINK™ NHS(N-Hydroxysuccinimidobiotin) chemistries according to the manufacturer's instructions (Pierce; Rockford, Ill.). For in vitro and in vivo experiments, lyophilized peptides were respectively dissolved in Dulbecco's Minimum Essential Media (DMEM, Gibco BRL; Grand Island, N.Y.) supplemented with 5% (V/V) FBS (Hyclone; Logan, Utah), 2 mM L-glutamine and 2% penicillin/streptomycin (Gibco BRL); or reconstituted in PBS and used at the indicated concentrations. The peptides were greater than 90% pure. The anti-fusion inhibitor, RFI-641 (Raznikov et al., 2001) was diluted in PBS and administered in vivo at 25 μg/dose.

Virus stocks. Wild type RSV strains A2 and B1 (Wright et al., 1973) and mutant RSV strains cpts248/404 (Ackerlind et al., 1988), rA2 cpts248/404ASH (Crowe et al., 1994) and cp32/D1, deficient in SH and G genes (nucleotides 4064-5462) (Karron et al., 1997), were propagated as previously described (Hancock et al., 1996) in HEp-2 cells (ATCC CCL 23) cultured in complete medium (DMEM supplemented with 2 mM L-glutamine, 2% penicillin/streptomycin and 10% (V/V) FBS, 37° C., 5% CO₂). The cp32/D1 mutant was isolated from the B1 strain using methodologies previously described for cp52 (Crowe et al., 1996; Karron et al., 1997). A recombinant RSV, (rA2 cpΔG118) genetically truncated at amino acid 117 of G protein was also used in the investigation. All virus stocks were prepared from cell lysates clarified by low speed (200 g) centrifugation 15 minutes at 4° C.

Inhibition of virus infectivity in vitro. HEp-2 cell monolayers were grown in 96-well tissue culture plates (Falcon, Becton Dickson and Co.; Franklin Lakes, N.J.) with complete medium. The monolayers were pre-treated for 1 hour at 4° C. or 37° C. in triplicate wells with the indicated amounts of recombinant chemokine or anti-chemokine receptor antibody. After removal of chemokine or antibody, the monolayers were infected 1 hour at the same temperature with the denoted wild type or mutant RSV strain and then overlaid with 2% Sephadex (Amersham Biosciences; Piscataway, N.J.). Anti-viral activity was also assessed 1 hour after the cells were infected, or following simultaneous exposure of the monolayers to virus in a 1:1 mixture with the indicated doses of chemokines or CCL5 peptides. The inhibitory properties of the anti-chemokine receptor antibodies were tested alone or in combination at doses ranging from 5 to 100 μg/ml culture medium.

Inhibition of virus infectivity in vivo. Female BALB/c mice (8-10 week old, Charles River Laboratories; Wilmington, Me.) were administered peptide (125 μg to 500 μg/dose) 1 hour before or 1 hour after challenge, or simultaneously in a 1:1 mixture with the A2 strain of RSV (˜2×10⁶ pfu). All administrations were intranasal (0.05 ml) and performed under injected anesthesia (60 mg ketamine/kg and 2.5 mg xylazine/kg (The Butler Co.; Dublin Ohio)). Four days later the lungs were removed, homogenized, clarified by low speed centrifugation, snap frozen, and stored at −70° C. until plaque assays were performed using HEp-2 cell monolayers (Hancock et al. 1996). All animals were housed in a facility accredited by the American Association for Accreditation of Laboratory Animal Care.

Plaque assay. Virus plaques were visualized as previously described (Hancock et al. 1996) using anti-F protein (L4) mAb (Paradiso et al., 1991; Walsh and Hruska, 1983). Thereafter, cells were rinsed with blotto blocking agent (PBS, 5% powdered milk) and incubated with horseradish peroxidase-conjugated goat anti-mouse IgG (Kirkegaard and Perry Laboratories, Inc.; Gaithersburg, Md.). Bound antibody was detected using 0.05% 4-chloro-1-naphtol containing 0.1% H₂O₂ in PBS. After enumeration of plaques, the mean percent infectivity (±1 standard deviation of triplicate wells) was calculated relative to monolayers exposed to virus in culture medium alone without chemokine or anti-chemokine receptor antibody.

Interaction of CCL5 peptides with HEp-2 cells. An ELISA was used to assess the relative binding of synthetic peptides to HEp-2 cell monolayers. In brief, 96-well plates (Falcon) were seeded with 3×10⁴ HEp-2 cells per well in 0.2 ml of complete medium. After overnight incubation (37° C., 5% CO₂), the monolayers were cooled on ice for approximately 15 minutes. The biotinylated peptides were added to duplicate wells in ascending doses from 0.49 μg to 500 μg per ml DMEM (supplemented with 1% FBS, 2% penicillin/streptomycin, and 2 mM L-glutamine) and incubated at 4° C. for 1 hour. The monolayers were then fixed (20 minutes at RT) with 80% methanol (J T Baker; Phillipsburg, N.J.). After washing with PBS, the monolayers were incubated (30 minutes at RT) with 0.3% (VN) H₂O₂ (Sigma; St. Louis Mo.), washed again, and probed (30 minutes at 4° C.) with biotinylated anti-human CCL5 mAb (R&D systems). Thereafter, the monolayers were incubated (1 hour at RT) with streptavidin-HRP (Zymed; San Francisco, Calif.). The wells were developed with TMB One-Step Substrate Solution (DAKO; Carpinteria, Calif.). Optical density was determined (450 nm, reference at 550 nm) with a Molecular Devices Versamax microplate reader (Sunnyvale, Calif.).

Flow Cytometry. To ascertain CCR expression, A549 and HEp-2 cells were gently removed from 6-well plates (Falcon) using a cell scraper and incubated (30 minutes at 4° C.) with biotinylated mAb against CCR1 (catalogue No. FAB182F), CCR3 (catalogue No. FAB145P), or CCR5 (catalogue No. FAB155P), followed thereafter by streptavidin-phycoerythrin. The cells were analyzed using standard flow cytometric techniques (FACSort, Becton Dickinson; Mountain View, Calif.). All reagents were purchased from R&D systems.

Detection of CCL5. An ELISA was used according to the manufacturer's (R&D Systems) instructions to detect secreted CCL5 in culture-supernatants of HEp-2 and A549 epithelial cells 24 hours after infection with RSV. Optical density was determined (450 nm, reference at 550 nm) with a Molecular Devices Versamax microplate reader. For determination of the number of CCL5 mRNA copies and RSV genomes following infection, quantitative polymerase chain reaction (qPCR) assays were developed. Total cellular RNA was isolated from cell monolayers using an RNeasy Mini Kit (Qiagen; Valencia, Calif.) and QiaShredders (Qiagen). RNA concentrations were measured on a CytoFluor® 4000 fluorescence plate reader (Applied Biosystems) after addition of RiboGreen RNA Quantitation Reagent (Molecular Probes; Eugene, Oreg.). One microgram of total cellular RNA was reverse transcribed using a TaqMan® Reverse Transcriptase Reagents kit (Applied Biosystems) according to manufacturer's specifications. The resulting cDNA were assayed for the presence of RSV genomes using a DNA primer-probe set complimentary to a region of the L gene from RSV A2. Additionally, the human RANTES TaqMan® Pre-Developed Assay Reagent (PDAR) (Applied Biosystems; Foster City, Calif.) containing the primer-probe set was used to detect CCL5 cDNA according to the manufacturer's specifications. PCR, fluorescence detection, and data analysis were performed on an ABI Prism 7700 Sequence Detector (Perkin-Elmer; Pittsburgh, Pa.).

Statistical Analyses. Significant differences (p<0.05) were determined after log transformation by Tukey-Kramer HSD multiple comparison or Student's t-test using JMP® statistical software (SAS Institute Inc.; Cary, N.C.). The data are expressed ±1 standard deviation. All data were confirmed in separate studies with similar results.

### Example 2

### CCL5 Inhibits Infection of Epithelial Cells with RSV

In this example, it was demonstrated in vitro that human airway epithelial cells produced CCL5 polypeptide in response to infection with RSV. Table 8 and Table 9 show representative results from experiments that respectively examined the effects of RSV infection on quantitative increases in mRNA copy number and secretion of CCL5 from A549 and HEp-2 cell monolayers. In A549 monolayers, CCL5 mRNA copy number increased two days (seven fold) and three days (twenty fold) after infection (Table 8). The data further indicated that as RSV replication progressed, the number of CCL5 transcripts dramatically increased relative to RSV genome copy number (Table 8). The ratio of CCL5 mRNA to RSV genome copy number increased sixteen fold between days one and three of culture. CCL5 protein was detected in culture supernatants as early as 24 hours after infection (Table 9). Thus, in response to RSV infection, the monolayers secreted increased amounts of CCL5 polypeptide.

TABLE 8
INFECTION OF HUMAN LUNG EPITHELIAL CELL LINES
WITH RSV INDUCES THE EXPRESSION OF CCL5^{a}

| | CCL5 Copy #/ng RNA^{b} | | CCL5 Copy #:RSV Genome^{c} | |
|---|---|---|---|---|
| Days | RSV | Control | RSV | Control |
| 1 | 6,423 | 16 | 11 | 0 |
| 2 | 43,507 | 12 | 62 | 0 |
| 3 | 126,660 | 8 | 180 | 0 |

^{a}A549 cells were infected (MOI = 0.09) with RSV A2 (RSV) or cultured in medium alone (Control) and CCL5 mRNA copy numbers and negative strand RSV genome copy numbers were determined on total cellular RNA by quantitative real-time PCR 1 to 3 days thereafter.
^{b}Denotes the number of CCL5 mRNA copies per ng total RNA.
^{c}Denotes the ratio of CCL5 mRNA copy numbers to RSV genome copy numbers.

TABLE 9
HUMAN EPITHELIAL CELL LINES INFECTED WITH RSV
SECRETE INCREASED AMOUNTS OF CCL5 INTO THE
CULTURE MEDIUM^{a}

| | |
|---|---|
| A549 | HEp-2 |

| Days | RSV | Control | RSV | Control |
|---|---|---|---|---|
| 1 | 447^{b} | 16 | 132 | 26 |
| 2 | 22,341 | 16 | ND^{c} | ND |
| 3 | 484,486 | 16 | ND | ND |

^{a}A549 and HEp-2 cells were infected (MOI = 0.09) with RSV A2 (RSV) or cultured in medium alone (Control). Culture supernatants were collected 1-3 days thereafter and analyzed for secreted CCL5 by ELISA.
^{b}The numbers represent pg CCL5 per ml culture medium.
^{c}ND denotes not determined.

The potential of CCL5 to inhibit infection of epithelial cells with RSV was further investigated. Table 10 and Table 11 depict separate experiments where prior exposure (1 hour) of HEp-2 cell monolayers with ascending amounts of human rCCL5 diminished infection. At the greatest dose tested (10 μg rCCL5/ml) infectivity was reduced to approximately 30% (Table 10) and 20% (Table 11) relative to control monolayers cultured in medium alone. The inhibitory effect equated to a median inhibitory concentration (IC₅₀) of 726 nm. Inhibition was dependent on dose, as 1 μg and 5 μg were less inhibitory than 10 μg rCCL5/ml culture medium. Prior exposure (1 hour) of HEp-2 cells to similar concentrations of recombinant MIP-1α/CCL3, MIP-1/CCL4, MCP-2/CCL8, or eotaxin/CCL11 (Table 10 and Table 11), or recombinant MIP-16/CCL15 or SDF-1α/CXCL12 (Table 12) did not impair infectivity. The data further indicated that inhibition of infectivity required treatment of cell monolayers with rCCL5 (10 μg/ml) prior to (Table 12), or at the same time virus was added to the monolayers (Table 13). rCCL5 did not reduce infectivity in vitro when administered 1 hour after virus adsorption (Table 12), or after heat denaturation (Table 13). Prior treatment of A549 monolayers with rCCL5 also inhibited infection with RSV (data not shown).

TABLE 10
CCL5 INHIBITS INFECTION OF HUMAN EPITHELIAL
CELLS WITH RSV^{a}

| | Percent Infectivity | | | |
|---|---|---|---|---|
| Dose (μg) | CCL5 | CCL3 | CCL8 | CCL11 |
| 10 | 29.2 ± 16.7 | 86.1 ± 2.4 | 94.4 ± 10.5 | 94.4 ± 4.8 |
| 5 | 25.0 ± 7.2 | 97.2 ± 8.7 | 100.0 ± 0.0 | 93.1 ± 12.7 |
| 1 | 73.6 ± 20.6 | 95.8 ± 11.0 | 88.9 ± 6.4 | 94.4 ± 4.8 |
| ^{a}HEp-2 cell monolayers were exposed to 1, 5, or 10 μg/ml of CCL5 (RANTES), CCL3 (MIP-1α), CCL8 (MCP-2), or CCL11 (eotaxin) for 1 hour. Thereafter, the monolayers were rinsed 3 times with medium and infected for 1 hour with the A2 strain of RSV. After 3 days incubation, plaques were enumerated to determine the degree of viral infectivity. The data are presented, as the mean percent infectivity (±1 standard deviation) relative | | | | |
| # to control wells (100% infectivity) incubated with virus in medium alone and not exposed to chemokine. | | | | |

TABLE 11
CCL5 INHIBITS INFECTION OF HUMAN EPITHELIAL CELLS
WITH RSV^{a}

Percent Infectivity

| Dose (μg) | CCL5 | CCL3 | CCL4 |
|---|---|---|---|
| 10 | 16.0 ± 8.0 | 91.0 ± 5.0 | 98.0 ± 5.0 |
| 5 | 42.0 ± 3.0 | 98.0 ± 1.0 | 99.0 ± 9.0 |
| 1 | 101.0 ± 4.0 | 103.0 ± 3.0 | 104.0 ± 4.0 |
| ^{a}HEp-2 cell monolayers were exposed to 1, 5, or 10 μg/ml of CCL5 (RANTES), CCL3 (MIP-1α) or CCL4 (MIP-1β) for 1 hour. Thereafter, the monolayers were rinsed 3 times with medium and infected for 1 h with the A2 strain of RSV. After 3 days incubation plaques were enumerated to determine the degree of viral infectivity. The data are presented, as the mean percent infectivity (±1 standard deviation) relative to control wells | | | |
| # (100% infectivity) incubated with virus in medium alone and not exposed to chemokine. | | | |

TABLE 12
INHIBITION OF INFECTION WITH RSV IS DEPENDENT
UPON PRIOR ADMINISTRATION OF CCL5^{a}

Percent Infectivity

| Treatment | CCL5 | CCL3 | CCL15 | CXCL12 |
|---|---|---|---|---|
| PRE | 22 ± 11 | 91 ± 10 | 97 ± 8 | 88 ± 7 |
| POST | 101 ± 12 | 104 ± 7 | 93 ± 4 | 104 ± 4 |

^{a}HEp-2 cell monolayers were treated with 10 μg/ml CCL5, CCL3, CCL15 (MIP-1δ) or CXCL12 (SDF-1) 1 hour prior to infection (PRE) or 1 hour after removal (POST) of RSV A2. Plaques were enumerated 3 days thereafter and presented as percent infectivity (±1 standard deviation) relative to control wells (100% infectivity) incubated with virus in medium alone and not exposed to chemokine.

TABLE 13
INHIBITION OF INFECTION IS DEPENDENT UPON PRIOR OR
SIMULTANEOUS ADMINISTRATION OF CCL5 WITH RSV
AND SENSITIVE TO HEAT DENATURATION^{a}

| | Percent Infectivity | | |
|---|---|---|---|
| CCL5 (μg) | PRE | SIM | HEAT |
| 10 | 17 | 33 | 75 |
| 0 | 83 | 89 | 94 |

^{a}CCL5 (10 μg/ml) or and equal volume of PBS were administered 1 hour prior to infection (PRE) or simultaneously (SIM) admixed with RSV to HEp-2 cell monolayers. Additional groups included CCL5 denatured by heat (HEAT). Plaques were enumerated 3 days thereafter.

### Example 3

### CCL5 Blocks the Interaction Between the Epithelial Cell and the Fusion (F) Protein of RSV

To ascertain whether inhibition of infection occurred directly by blocking virus interactions with cell surface ligands, or secondarily to outside in signaling pathways following ligand-receptor interactions, experiments were performed with rCCL5 that retained the N-terminal methionine (met)-CCL5 (Proudfoot et al., 1996). Met-CCL5 retains the initiating methionine and upon receptor binding is not biologically active (Simmons et al., 2000). At the greatest dose tested (20 μg met-CCL5/ml) infectivity was approximately 25% of control cells (FIG. 1). The inhibitory properties of met-CCL5 were also dependent on dose. Prior treatment with 1.25 μg met-CCL5/ml resulted in approximately 80% infectivity, while doses of 0.313 or 0.156 μg met-CCL5/ml were not inhibitory. Inhibition of replication was also tested when both infection and administration of rCCL5 were performed sequentially at 4° C. to impede CCR aggregation, which is important for outside in signaling (Blanpain et al., 2002). Infectivity following pre-treatment (1 hour) at 4° C. with 20 μg rCCL5/ml was approximately 14% of control and similar (16%) to that observed when the monolayers were sequentially treated with rCCL5 and infected at 37° C. (Table 14). Thus, the data suggested that rCCL5 could inhibit replication under conditions (4° C.) that would limit an intracellular signaling cascade.

TABLE 14
PRIOR TREATMENT OF HUMAN EPITHELIAL CELL LINES
WITH CCL5 AT 4° C. OR 37° C. INHIBITS INFECTION
WITH RSV^{a}

Percent Infectivity

| CCL5 (μg) | 4° C. | 37° C. |
|---|---|---|
| 20 | 14 | 16 |
| 10 | 23 | 4 |
| 5 | 37 | 41 |

^{a}HEp-2 cell monolayers were pre-treated for 1 hour at 4° C. or 37° C. with the indicated doses of recombinant CCL5. Plaques were enumerated 3 days after infection with RSV A2 and presented as percent infectivity relative to control wells (100% infectivity) incubated with virus in medium alone and not exposed to chemokine.

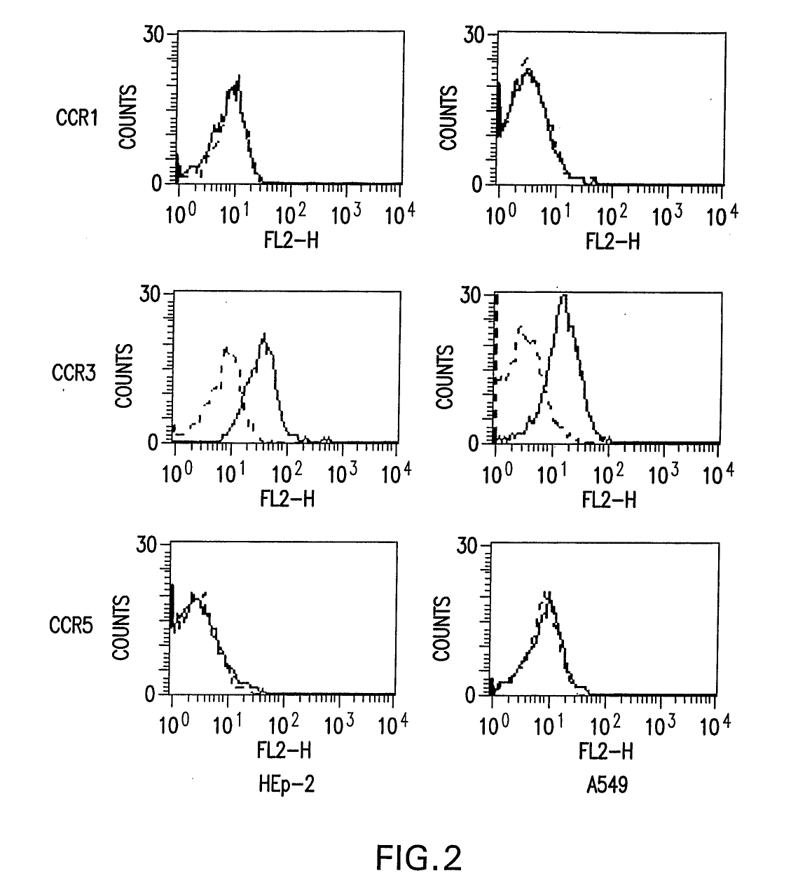

It was recently reported that activated bronchial epithelial cells express CCR3 (Stellato et al., 2001). Because CCL5 is also a ligand for CCR1 and CCR5 (Pakianathan et al., 1997) epithelial cells were examined by flow cytometry for receptors known to bind CCL5. The results depicted in FIG. 2 confirmed that CCR3 was expressed on the surface of HEp-2 and A549 epithelial cells. CCR1 and CCR5 were not detected. Thus, the data indicated that CCL5 blocks interactions between RSV and CCR3 on the epithelial cell surface. Additional recombinant chemokines known to bind CCR3 (Baggiolini, 2001) were tested to determine whether they too reduced infectivity. Prior treatment of HEp-2 cell monolayers with ascending amounts of recombinant eotaxin/CCL11, MCP-2/CCL8, or MIP-16/CCL15 did not impair RSV infectivity (Table 10 and Table 12). Because CXCR4 is also functionally expressed on activated bronchial epithelial cells (Eddleston et al., 2002) recombinant SDF-1δ/CXCL12 was also tested, but did not impair infection (Table 12). Pre-incubation of HEp-2 cell monolayers or infection in the presence of poly or monoclonal anti-chemokine receptor antibodies did not reduce RSV infectivity (data not shown).

Mechanism(s) of inhibition were further investigated by examining the ability of rCCL5 to inhibit infection by RSV strains deficient in one, or two of the 3 glycoproteins (F G, and SH proteins) located in the virus envelope. A series of studies were performed using genetically modified strains deleted of the SH protein (rA2 cpts248/404ΔSH), or with the C-terminal ectodomain of G protein truncated at amino acid 118 (rA2 cpGΔ118). Table 15 demonstrates that pretreatment with 10 μg/ml of rCCL5 or met-CCL5 reduced the infectivity of rA2 cpGΔ118 and rA2 cpts248/404ΔSH viruses relative to control cells cultured with virus in medium alone. Prior treatment with rCCL5 (10 μg/ml) also reduced infection by mutant cp32/D1 (lacking both SH and G proteins) and parent B1 strains of RSV (Table 8). Thus, rCCL5 inhibited infection by viruses deficient in G and/or SH proteins. Taken together, the data indicated that rCCL5 blocked interactions that occurred between F protein in the envelope and the epithelial cell surface.

TABLE 15
CCL5 INHIBITS MUTANT RSV STRAINS LACKING THE SH
AND/OR G ENVELOPE GLYCOPROTEINS FROM INFECTING
HEP-2 CELLS^{a}

| Percent Infectivity | |
|---|---|
| Experiment 1 | Experiment 2 |

| Strain | CCL5 | met-CCL5 | CCL5 | met-CCL5 |
|---|---|---|---|---|
| 248/404ΔSH | 34 | 32 | 13 | ND^{b} |
| 248/404 | 28 | 26 | 25 | ND |
| cp32/D1 | ND | ND | 41 | ND |
| rA2cpΔ118 | 26 | 41 | ND | ND |
| cp-RSV | 64 | 64 | ND | ND |
| B1 | ND | ND | 24 | ND |
| A2 | 33 | 31 | 28 | ND |
| ^{a}HEp-2 cell monolayers were treated 1 h with 10 μg/ml recombinant CCL5 or met-CCL5. After removal of CCL5, the monolayers were infected with the indicated RSV strains. Experiment 1 and Experiment 2 denote the results from 2 separate studies. Plaques were visualized after 3 (A2) or 5 days (rA2cpΔ118, 248/404, 248/404ΔSH, B1, and cp32/D1) incubation. The data are presented as percent infectivity relative to control wells | | | | |
| # (100% infectivity) incubated with virus in medium alone and not exposed to chemokine. | | | | |

^{b}ND denotes not done.

### Example 4

### RSV Infection is Inhibited In Vivo by a Synthetic N-Terminal Peptide of CCL5

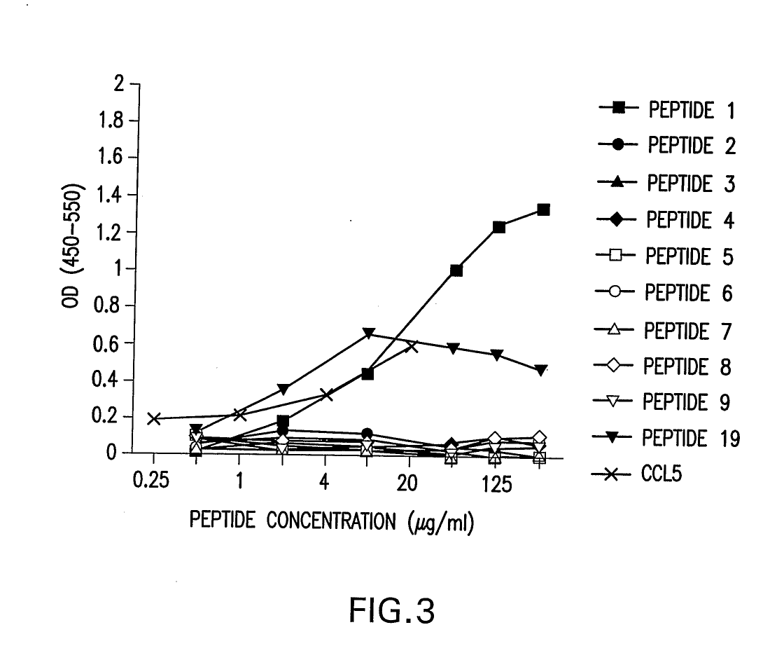

CCL5 may inhibit infection by blocking interactions between F protein in the envelope and chemokine receptor (e.g. CCR3) or negatively charged glycosaminoglycan (GAG) on the epithelial cell surface. F protein has a heparin-binding (HBD) motif composed of positively charged amino acids that is important for infection of airway epithelial cells (Feldman et al., 2000). The C terminal α-helical region of CCL5 was shown to play a role in inhibiting HIV-1 infection (Burns et al., 1998). The amino acid residues that interact with cognate receptor or GAG are respectively located in the N-terminal (Pakianathan et al., 1997) and C-terminal (Proudfoot et al. 2001) regions of CCL5. To examine these possibilities, a series of peptides (15-mers, overlapping by 7 amino acids) representing all 68 amino acids of CCL5 were synthesized (Table 2). As an initial screen, the peptides were biotinylated and assessed for binding to human epithelial cells. FIG. 3 depicts a representative experiment wherein increasing concentrations of biotinylated peptides were incubated with viable HEp-2 cells at 4° C. The resultant data indicated that rCCL5 and peptide 1 (SEQ ID NO:2), representing the N-terminal 15 residues of CCL5 readily bound to the epithelial cell monolayer. A synthetic peptide (peptide 19) containing the consensus HBD of RSV G protein (Feldman et al., 1999) also bound to the monolayer. Peptides 7-9, which represented the HBD of CCL5, did not readily bind to the monolayer. Thus, the data indicated that inhibition of infectivity occurred because the N-terminal region of CCL5 (peptide 1; SEQ ID NO:2) blocked interactions between F protein in the envelope and the epithelial cell surface.

To test the hypothesis further, the same peptides of CCL5 were assessed in the BALB/c mouse model for inhibition of infection by the A2 strain of RSV. The results depicted in Table 16 and Table 17 are representative of five experiments. The results demonstrated that peptide 1 (500 μg/dose, 10 mg/ml) was inhibitory in vivo when administered simultaneously with virus (Table 16 and Table 17) or given 1 hour prior to infection (Table 17). The lungs of naïve BALB/c mice four days after infection contained approximately 5 log₁₀ PFU per gram of tissue. In comparison, RSV titers in the lungs of mice co-administered peptide 1 were 1,000 (Table 16 and Table 17) to 100-fold (Table 16) less. When peptide 1 was administered 1 hour before infection, virus load was diminished more than 50-fold, and significantly less than that observed in naïve mice (Table 17). The average reduction in virus load associated with the administration of 300 μg or more of peptide 1 in five experiments was 2.4 log₁₀. The inhibitory properties of peptide 1 were dependent on dose. Co-administration of 300-500 μg peptide 1 significantly reduced virus load. This was not observed with 250 μg or 125 μg doses (Table 16). Similar to in vitro studies with rCCL5 (Table 12), peptide 1 did not inhibit infection when administered 1 hour after infection (Table 17).

The data suggested that peptides representing the C-terminal HBD (peptides 7-9) of CCL5 (500 μg/dose, 10 mg/ml) were less inhibitory. The data presented in Table 16 demonstrates that co-administration of peptides 8 or 9 with RSV reduced infectivity (approximately 10 fold). The average reduction in virus load associated with peptides 7-9 for all experiments at the 500 μg/dose was respectively 1.1, 1.0, and 1.4 log₁₀. The average reduction in virus load associated with peptides 2, 4, and 6 for all experiments at the 500 μg/dose was respectively 0.9, 1.2, and 0.5 log₁₀ (data not shown). Peptide 19, representing amino acids 184-198 and the HBD of RSV G protein, and the pharmaceutical compound RFI-641 (Razinkov et al., 2001) were inhibitory against RSV infection. Thus, amongst the peptides of CCL5, peptide 1 bound viable epithelial cells in vitro and was most inhibitory in vivo. Inhibition by all peptides in vitro occurred only at IC₅₀ values from 391 to 525 μM (data not shown).

TABLE 16
THE ANTI-VIRAL ACTIVITY OF SYNTHETIC PEPTIDES OF
CCL5 IN VIVO^{a}

RSV/Gram Lung Tissue (log₁₀)

| Peptide (μg) | Exp. 1 | Exp. 2 |
|---|---|---|
| #1 (500) | 1.8 ± 0.1 | 3.1 ± 0.1 |
| #1 (400) | ND | 3.8 ± 0.1 |
| #1 (300) | ND | 4.0 ± 0.1 |
| #1 (250) | 4.3 ± 0.2 | ND |
| #1 (125) | 4.6 ± 0.2 | ND |
| #7 (500) | 4.6 ± 0.1 | ND |
| #8 (500) | 3.6 ± 0.03 | ND |
| #9 (250) | 3.7 ± 0.1 | ND |
| #19 (500)^{b} | 1.8 ± 0.1 | 1.8 ± 0.04 |
| RFI-641 (25)^{b} | 2.7 ± 0.2 | 2.7 ± 0.3 |
| PBS^{b} | 4.7 ± 0.02 | 5.2 ± 0.1 |

^{a}BALB/c mice were simultaneously administered RSV A2 (˜1 × 10⁶ PFU) admixed in equal volume with the indicated amounts (μg/dose) of synthetic peptides. Four days thereafter, geometric mean infectious virus titers (±1 standard deviation) were determined in the lungs. The limit of detection of the assay was approximately 1.5 log₁₀. There were 5 mice per group.
^{b}Control mice were administered RSV admixed with the indicated amounts of peptide #19 (representing the heparin binding domain of RSV G protein), RFI-641, or PBS.

TABLE 17
PEPTIDE 1 OF CCL5 IS INHIBITORY IN VIVO WHEN
GIVEN BEFORE, BUT NOT AFTER INFECTION WITH RSV

| Peptide (μg) | Treatment^{a} | RSV/Gram Lung Tissue (log₁₀) |
|---|---|---|
| #1 (500) | Simultaneous | 1.9 ± 0.2 |
| #1 (500) | Pre | 3.5 ± 0.6 |
| #1 (500) | Post | 5.3 ± 0.2 |
| #7 (500) | Simultaneous | 3.8 ± 0.1 |
| PBS | Simultaneous | 5.4 ± 0.1 |
| ^{a}BALB/c mice were administered 500 μg of the indicated CCL5 peptide 1 hour before (Pre) or 1 hour after (Post) infection, or admixed in equal volume and administered simultaneously (Simultaneous) with RSV A2 Control mice were administered an equivalent dose (˜1 × 10⁶ PFU) of RSV admixed in equal volume with peptide #7 or PBS. Four days thereafter, geometric mean infectious virus titers (±1 standard deviation) were determined. | | |
| # The limit of detection of the assay was approximately 1.5 log₁₀. There were 5 mice per group. | | |

### Example 5

### Comparison of CCL5 and CCL3 Polypeptide Sequences

As detailed in Example 2, the chemokine CCL5, but not CCL3 (MIP-1α) or CCL4 (MIP-1β) inhibit RSV infection of epithelial cells. This indicated that RSV utilizes a receptor other than CCR5. Thus, to further elucidate the sequence and/or structural requirements of CCL5 mediated inhibition of RSV infection, CCL5 and CCL3 polypeptides were compared by amino acid sequence alignment (Table 6), hydropathy plots (FIG. 4A and FIG. 4B) and molecular modeling/visualization (data not shown).

The amino acid sequences of CCL5 (SEQ ID NO:1) and CCL3 (SEQ ID NO:21) were first compared via the gapped “BLAST 2 Sequences” alignment algorithm (BLASTP version 2.2.6; Default Matrix BLOSUM62), which is an interactive tool that utilizes the BLAST engine for pairwise protein-protein (or DNA-DNA) sequence comparison and generates a gapped alignment by using dynamic programming to extend the central pair of aligned residues (Tatusova and Madden, 1999). The CCL5 amino acid sequence is presented above the BLASTP alignment (boxed residues) and the CCL3 amino acid sequence is presented below the alignment (Table 6). The BLAST alignment of CCL5 (amino acid residues 4-68) and CCL3 (amino acid residues 3-69) indicates that these polypeptides share 32 identical amino acids (i.e., 48% sequence identity) and have about 78% amino acid sequence similarity. The alignment includes a one amino acid gap between Tyr7 and Tyr8 of CCL5 (SEQ ID NO:1), the omission of the first three NH₂-terminal amino acids of CCL5 (Ser1-Tyr3 of SEQ ID NO:1), the omission of the first two NH₂-terminal amino acids of CCL3 (Ser1-Leu2 of SEQ ID NO:21) and the omission of the COOH-terminal amino acid of CCL3 (Ala69 of SEQ ID NO:21).

A comparison of the hydropathy plots (Kyte and Doolittle, 1982) of full length CCL5 versus full length CCL3 (FIG. 4A and FIG. 4B) indicated that the greatest hydropathic sequence divergence between CCL5 and CCL3 occurs within NH₂-terminus of these polypeptides. For example, the first four NH₂-terminal amino acids of CCL5 (Ser1-Pro2-Tyr3-Ser4) are hydrophilic whereas the first four NH₂-terminal amino acids of CCL3 (Ser1-Leu2-Ala3-Ala4) are predominantly hydrophobic (data not shown). The hydropathy score for the first four amino acids of CCL5 and CCL3 could not be calculated with a sliding window size of nine, and as such, these amino acids are omitted from the hydropathy plots shown in FIG. 4A and FIG. 4B. The NH₂-terminus of CCL5 remains hydrophilic up to about amino acid eight (Ser8), which is then followed by hydrophobic amino acids Pro9-Cys10-Cys11-Phe12-Ala13-Tyr14-Iso15-Ala16 of NH₂-loop structure. Interestingly, the NH₂-terminus of CCL3, up to about amino acid thirty, has an inverse relationship with (or mirrors) the hydropathy profile of CCL5. In contrast, from about amino acid thirty-one to the end of the COOH-terminus, the hydropathy profiles of these two polypeptides are nearly identical (FIG. 4A).

The energy minimized structures of CCL5 (PBD 1RTO; Skelton et al., 1995) and CCL3 (PBD 1B53; Czaplewski et al., 1999) were modeled using the publicly available (i.e., accessible via the ExPASy world wide web server) software package SWISS-MODEL and Swiss-PdbViewer (Guex and Peitsch, 1997). The two proteins' tertiary structures (or fold) superimposed with the exception of the NH₂-terminal amino acids 1 to 7 and COOH-terminal amino acids 64 to 68 (data not shown). A best fit analysis (SWISS-MODEL “Magic Fit”) of a carbon backbone trace (drawn as ribbon diagram) of the full length CCL5 polypeptide (amino acids Ser1 to Ser68 of SEQ ID NO:22). Similar to the hydropathy plots, the greatest structural sequence divergence between CCL5 and CCL3 occurs in the NH₂-terminal amino acid residues.

Finally, the NH₂-terminal portion of the CCL5 protein comprising the peptide 1 fragment (i.e., amino acids 1 to 15), which was demonstrated to be the most inhibitory against RSV infection, was structurally analyzed (data not shown). The dihedral angles (Phi and Psi) of CCL5, Met-CCL5 and AOP-CCL5 were calculated in silico (SWISS-MODEL and Swiss-PdbViewer) using the Brookhaven Protein Data Bank molecular coordinates, deposited as PDB names 1RTO, 1EQT and 1B3A, respectively. The results of these calculations are shown in Table 7.

### Example 6

### Chemokine Agonist Assays

Chemotaxis Assays. THP-1 cell chemotaxis (cell migration) is carried out according to the method of Gong and Clark-Lewis (1995) as modified by Proudfoot et al. (1996). Briefly, 5.6×10⁵ cells in 200 μl of medium (RPMI 1640 containing 0.01 M HEPES, 10% heat-inactivated fetal calf serum, 2 mM L-glutamine, and 0.005% gentamicin) are placed in the upper chambers of a 96-well Boyden microchamber (NeuroProbe; Cabin John, Md.) fitted with 5-μm filters. Subsequently, 370 μl of the medium described above (minus the fetal calf serum), containing the ligand and appropriate dilutions of Met-CCL5, are placed in the lower chambers of the 96-well Boyden microchamber.

After sixty minutes of incubation at 37° C. under 5% CO₂, the cells are removed from the upper wells, and 200 p1 of phosphate-buffered saline containing 20 μM EDTA added to detach the cells bound to the filter. After thirty minutes of incubation at 4° C., the plate is centrifuged at 1800×g for ten minutes, and the supernatants are removed from the lower wells. The number of cells that migrate are measured by the Cell Titer 96™ non-radioactive cell proliferation assay (Promega), which monitors the conversion of tetrazolium blue into its formazan product.

Monocytes and neutrophil chemotaxis is measured as described by Fincham et al. (1988). Briefly, 50 ml of fresh blood is collected into a 15-ml solution containing 0.1 M EDTA, 3% Dextran and 3% glucose to prevent aggregation. This mixture is allowed to sediment for one hour at 37° C. The PMNs and lymphocytes are separated by layering 14 ml of plasma onto 7 ml of Ficoll and centrifuging for twenty minutes at 296×g and 15° C. with the centrifuge brake off. The lymphocytes are located at the interface of the Ficoll and the plasma, whereas the PMNs form a pellet. Contaminating erythrocytes are removed from the PMNs (mainly neutrophils) by hypotonic lysis, and residual leukocytes are washed and resuspended at a concentration of 106 leukocytes/ml in RPMI 1640 medium. Approximately 40-50×10⁶ monocytes/ml are purified from the lymphocyte fraction by adding 106 sheep red blood cells/ml and rosetting for sixty minutes at 4° C., followed by a further Ficoll gradient centrifugation. The monocytes are washed in PBS buffer (140 mM NaCl, 3 mM KCl, 8 mM Na₂HPO₄, 1.5 mM KH₂PO₄, pH 7.4) and resuspended in RPMI 1640 medium.

A 96-well Boyden microchamber is used to assay monocyte and neutrophil chemotaxis. Serial dilutions of the test antiviral molecule (e.g., a modified NH2-terminal CCL5 peptide fragment) is made in medium (RPMI 1640 with 2 mM L-glutamine, 25 mM HEPES, and 10% heat inactivated fetal calf serum).

Twenty-five μl of chemoattractant was added to the lower chamber of the assay wells and covered with a polyvinylpyrrolidone-free polycarbonate membrane with pore size of 3 μm for neutrophils and 5 μm for monocytes. A 50-μl solution containing 10⁶ cells/ml was then added to the top wells. The assay plates were incubated at 37° C. for twenty minutes for neutrophils and thirty minutes for monocytes. The upper surface of the membranes was then washed with PBS buffer, and the cells on the underside of the membrane were fixed in methanol. The membranes were stained with a mixture of Field's A and B stains (Bender and Hobein) and air-dried. The cells on the under surface of the membranes were then counted using a Zeiss Axiophot microscope and the VIDAS image analyzer software (KONTRON Electronics, Zurich, Switzerland).

Calcium Mobilization Assay. Mobilization of neutrophil intracellular calcium is measured with recombinant or synthetic CCL5 and test antiviral molecules over a concentration range of 10-10M. Cells are incubated in Krebs Ringer buffer (1.36 mM NaCl, 1.8 mM KCl, 1.2 mM KH₂PO₄, 1.2 mM MgSO₄, 5 mM NaHCO₃, 1.2 mM CaCl₂, 0.21 mM EGTA, 5.5 mM D-glucose, 20 mM HEPES) for thirty minutes at 37° C. with 2 μM Fura-2 dye.

The Fura-2 dye is excited at 340 nm and fluorescence emission is monitored at 500 nm using fluorometer. Intracellular Ca²⁺ is calculated using the equation, [Ca]=K_{d} (F−Fₘᵢₙ)/(Fₘₐₓ−F), where K_{d} is the dissociation constant for Ca binding to the dye and F is in arbitrary fluorescent units. An excess of 10 mM EGTA is added to chelate the Ca²⁺ and calculate Fₘᵢₙ. The pH is adjusted to 8.5 by adding 20 mM Tris and the cells are lysed with 50 μM digitonin. Fₘₐₓ is calculated from the fluorescence value after exposing the lysed cells to an excess of 1 mM Ca²⁺.

### REFERENCES

U.S. Pat. No. 5,258,454
U.S. Pat. No. 5,556,747
U.S. Pat. No. 5,789,166
U.S. Pat. No. 6,391,548
U.S. Pat. No. 5,817,879
Akerlind et al., “Respiratory syncytial virus: heterogeneity of subgroup strains”, J Gen Virol, 69: 2145-2154, 1988.
Altschul et al., “Basic local alignment search tool”, J. Mol. Biol., 215:403-410, 1990.
Appay and Rowland-Jones, “RANTES: a versatile and controversial chemokine”, Trends Immunol, 22:83-87, 2001.
Arenzana-Seisdedos et al., “HIV Blocked By Chemokine Antagonist”, Nature, 383:400, 1996.
Baggiolini, “Chemokines in pathology and medicine”, J Intern Med, 250:91-104, 2001.
Barany et al., Int. J. Peptide Protein Res. 30, 705-739, 1987.
Barron and Zuckermann, “Bioinspired Polymeric Materials: In-Between Proteins and Plastic”, Curr. Opin. Chem. Biol., 3:681-687, 1999.
Bartz et al., “Respiratory syncytial virus decreases the capacity of myeloid dendritic cells to induce interferon-gamma in naïve T cells”, Immunology, 109:49-57, 2003.
Beaulieu et al., “Expression of a functional eotaxin (CC chemokine ligand 11) receptor CCR3 by human dendritic cells”, J Immunol, 169:2925-2936, 2002.
Becker et al., “RSV infection of human airway epithelial cells causes production of the beta chemokine RANTES”, Am. J. Physiol, 272:L512-520, 1997.
Berger et al., “Chemokine receptors as HIV-1 coreceptors: Roles in viral entry, tropism and disease”, Annu Rev Immunol, 17:657-700, 1999.
Blanpain et al., “Multiple active states and oligomerization of CCR5 revealed by functional properties of monoclonal antibodies”, Mol. Biol. Cell, 13:723-737, 2002.
Bode and Huber, Eur. J. Biochem. 204:433-451, 1992.
Bonville et al., “Macrophage inflammatory protein-1-alpha and RANTES are present in nasal secretions during ongoing upper respiratory tract infection”, Ped Allergy and Immunology, 10:39-44, 1999.
Bourgeois et al., “Heparin-like structures on respiratory syncytial virus are involved in its infectivity in vitro”, J Virol, 72: 7221-7227, 1998.
Burns et al., “A new monoclonal antibody, mAb 4A12, identifies a role for the glycosaminoglycan (GAG) binding domain of RANTES in the antiviral effect against HIV-1 and intracellular Ca2+ signaling”, J Exp Med, 188:1917-1927, 1998.
Chung et al., “The Three-Dimensional Solution Structure of RANTES”, Biochemistry, 34:9307-9314, 1995.
Clark-Lewis et al., M. J. Biol. Chem., 269:16075-16081, 1994.
Crowe et al., “A further attenuated derivative of a cold-passaged temperature-sensitive mutant of human respiratory syncytial virus retains immunogenicity and protective efficacy against wild-type challenge in seronegative chimpanzees”, Vaccine, 12:783-790, 1994.
Crowe et al., “Live subgroup B respiratory syncytial virus vaccines that are attenuated, genetically stable, and immunogenic in rodents and nonhuman primates”, J Infect Dis, 173:829-839, 1996.
Crump et al., EMBO J., 16 6996-7007, 1997.
Czaplewski et al., “Identification of Amino Acid Residues Critical for Aggregation of Human Cc Chemokines Macrophage Inflammatory Protein (Mip)-1Alpha, Mip-1Beta, and Rantes. Characterisation of Active Disaggregated Chemokine Variants”, J. Biol. Chem. 274:16077, 1999.
Dairaghi et al., “Abduction of chemokine elements by Herpesviruses”, Seminars in Virology, 8:377-385, 1998.
Domachowske et al., “Respiratory syncytial virus infection: immune response, immunopathogenesis, and treatment”, Clin Microbiol Rev, 12:298-309, 1999.
Dorig et al., “The human CD46 is a receptor for measles virus (Edmonston strain)”, Cell, 75:295-305, 1993.
Dorner et al., “MIP-1alpha, MIP-1beta, RANTES, and ATAC/lymphotactin function together with IFN-gamma as type 1 cytokines”, Proc Natl Acad Sci USA. 99:6181-6186, 2002.
Eddleston et al., “Functional expression of the C—X—C chemokine receptor CXCR4 by human bronchial epithelial cells: regulation by proinflammatory mediators”, J Immunol, 169:6445-6451, 2002.
Eguchi and Kahn, “Design, Synthesis, and Application of Peptide Secondary Structure Mimetics, Mini Reviews in Medicinal Chemistry, 2:447-462, 2002.
Eisenberg et al., J. Mol. Biol., 179:125-142, 1984.
Elliott et al., “Recombinant respiratory syncytial viruses lacking the C-terminal third of the attachment (G) protein are immunogenic and attenuated in vivo and in vitro”, Manuscript submitted, 2004.
Fairlie et al., J. Med. Chem., 43:1271-1281, 2000.
Feldman et al., “Identification of a linear heparin binding domain for human respiratory syncytial virus attachment glycoprotein”, G. J. Virol., 73:6610-6617, 1999.
Feldman et al., “The fusion glycoprotein of human respiratory syncytial virus facilitates virus attachment and infectivity via an interaction with cellular heparin sulfate”, J Virol, 74:6442-6447, 2000.
Fincham et al., J. Immunol. 140, 4294-4299, 1988.
Gellman, “Foldamers: A Manifesto”, Acc. Chem. Res., 31:173-180, 1998.
Glen and Fairlie, “Mimetics of the Peptide β-Strand”, Mini Reviews in Medicinal Chemistry, 2:433-445, 2002.
Gong and Clark-Lewis, J. Exp. Med., 181:631-640, 1995.
Gorman et al., “Antiviral activity and structural characteristics of the nonglycosylated central subdomain of human respiratory syncytial virus attachment (G) glycoprotein”, J Biol. Chem., 276:38988-94, 2001.
Guex and Peitsch, “SWISS-MODEL and the Swiss-PdbViewer: An environment for comparative protein modeling”, Electrophoresis 18:2714-2723, 1997.
Hall C. B., “Respiratory syncytial virus and parainfluenza virus”, N Engl J Med, 21: 344:1917-28, 2001.
Hancock et al., “Generation of atypical pulmonary inflammatory responses in BALB/c mice after immunization with the native attachment (G) glycoprotein of respiratory syncytial virus”, J Virol, 70:7783-7791, 1996.
Harrison et al., “Respiratory syncytial virus-induced chemokine expression in the lower airways: eosinophil recruitment and degranulation”, Am J Resp Crit Care Med, 159:1918-1924, 1999.
Hemmerich et al., Biochemistry, 38:13013-13025, 1999.
Hickling et al., “A recombinant trimeric surfactant protein D carbohydrate recognition domain inhibits respiratory syncytial virus infection in vitro and in vivo”, Eur J Immunol, 29:3478-3484, 1999.
Hoover et al., “The Crystal Structure of Met-Rantes: Comparison with Native Rantes and Aop-Rantes”, Protein and Peptide Letters, 7:200, 2000.
Hopp and Woods, Proc. Natl. Acad. Sci. U.S.A., 78:3824-3828, 1981.
Jaovisidha et al., “Respiratory syncytial virus stimulates neutrophil degranulation and chemokine release”, J Immunol, 163:2816-2820, 1999.
Jois et al., “Design of β-turn Based Therapeutic Agents”, Current Pharmaceutical Design, 9:1209-1224, 2003.
Karron et al., “Respiratory syncytial virus (RSV) SH and G proteins are not essential for viral replication in vitro: clinical evaluation and molecular characterization of a cold-passaged, attenuated RSV subgroup B mutant”, Proc Nat Acad Sci USA, 94:13961-13966, 1997.
Kirshenbaum et al., “Designing Polymers that Mimic Biomolecules”, Curr. Opin. Struct Biol., 9:530-535, 1999.
Krilov L. R., “Palivizumab, in the prevention of respiratory syncytial virus disease”, Expert Opin Biol Ther, 2:763-769, 2002.
Krilov L R, “Safety issues related to the administration of ribavirin”, Pediatr Infect Dis J, 21:479-81, 2002.
Krusat et al., “Heparin-dependent attachment of respiratory syncytial virus (RSV) to host cells”, Arch\Virol, 142:1247-1254, 1997.
Kyte and Doolittle, J. Mol. Biol., 157:105-132, 1982.
Lalani et al., “Use of chemokine receptors by poxviruses”, Science, 286:1968-1971, 1999.
Langedijk et al., “Structural homology of the central conserved region of the attachment protein G of respiratory syncytial virus with the fourth subdomain of 55-kDa tumor necrosis factor receptor”, Virology, 243:293-302, 1998.
Laurence et al., Biochemistry, 39: 3401-3409, 2000.
Lee et al., “Epitope mapping of CCR5 reveals multiple conformational states and distinct but overlapping structures involved in chemokine and coreceptor function”, J. Biol. Chem., 274:9617-9626, 1999.
Lehner T., “The role of CCR5 chemokine ligands and antibodies to CCR5 coreceptors in preventing HIV infection”, Trends Immunol, 23:347-351, 2002.
Liu et al., “Cell surface heparan sulfate and its roles in assisting viral infections”, Med Res Rev., 22:1-25, 2002.
Locati et al., “Chemokines and chemokine receptors: biology and clinical relevance in inflammation and AIDS”, Annu. Rev. Med., 50:425-440, 1999. Lowman et al. J. Biol. Chem., 271:14344-14352, 1996.
Lusso P., “HIV and chemokines: implications for therapy and vaccine”, Vaccine, 20:1964-1967, 2002.
Malhotra et al., “Isolation and characterization of potential respiratory syncytial virus receptor(s) on epithelial cells”, Microbes Infect, 5:123-33, 2003.
Martin et al., “Structural and functional analysis of the RANTES-glycosaminoglycans interactions”, Biochemistry, 40:6303-6318, 2001.
Martinez et al., “Enhanced neutralization of human respiratory syncytial virus by mixtures of monoclonal antibodies to the attachment (G) glycoprotein”, J Gen Viro, 79:2215-2220, 1998.
Monfardini et al., “Structure-based Design of Mimetics fro GM-CSF”, Current Pharmaceutical Design, 8:2185-2199, 2002.
Moser et al., “Lymphocyte traffic control by chemokines”, Nat Immunol, 2:123-128, 2001.
Mosier et al., “Highly Potent RANTES Analogues Either Prevent CCR5-Using Human Immunodeficiency Virus Type 1 Infection In Vivo or Rapidly Select for CXR4-Using Variants”, J. of Virology, 73:3544-3550, 1999.
Nardese et al., “Structural determinants of CCR5 recognition and HIV-1 blockade in RANTES”, Nat Struct Biol, 8:611-615, 2001.
Nardese et al., “Structural determinants of CCR5 recognition and HIV-1 blockade in RANTES”, Nature Structural Biology, 8:611-615, 2001.
Noah et al., “Chemokines and inflammation in the nasal passages of infants with respiratory syncytial virus bronchiolitis”, Clin Immunol, 104:86-95, 2002.
Olsewska-Pazdrak et al., “Cell-specific expression of RANTES, MCP-1, and MIP-1alpha by lower airway epithelial cells and eosinophils infected with respiratory syncytial virus”, J. Virol, 72:4756-4764, 1998.
Pakianathan et al., “Distinct but overlapping epitopes for the interaction of a CC-chemokine with CCR1, CCR3 and CCR5”, Biochemistry, 36:9642-9648, 1997.
Pakianathan et al., Biochemistry 36: 9642-8648, 1997.
Paradiso, et al., “Mapping of a fusion related epitope of the respiratory syncytial virus fusion protein”, Vaccine, 9:231-237, 1991.
Pastey et al., “A RhoA-derived peptide inhibits syncytium formation induced by respiratory syncytial virus and parainfluenza virus type 3”, Nat Med, 6:35-40, 2000.
Patch and Barron, “Mimicry of Bioactive Peptides via Non-Natural, Sequence-Specific Peptidomimetic Oligomers”, Current Opin. In Chem. Biol., 6:872-877, 2002.
Proudfoot et al., “Extension of recombinant human RANTES by the retention of the initiating methionine produces a potent antagonist”, J Biol Chem, 271:2599-2603, 1996.
Proudfoot et al., “Strategies for chemokine antagonists as therapeutics. Semin Immunol., 15:57-65, 2003.
Proudfoot et al., “The BBXB motif of RANTES is the principal site for heparin binding and controls receptor selectivity”, J Biol Chem, 276:10620-10626, 2001.
Razinkov et al., “RFI-641 inhibits entry of respiratory syncytial virus via interactions with fusion protein”, Chem Biol., 8:645-659, 2001.
Saito et al., “Respiratory syncytial virus induces selective production of the chemokine RANTES by upper airway epithelial cells,” J. Infect. Dis, 175:497-504, 1997.
Schall et al., “A human T cell-specific molecule is a member of a new gene family”, J. Immunol, 141:1018-1025, 1988.
Schraufstätter et al., J. Biol. Chem., 270:10428-10431, 1995.
Simmons et al., “Co-receptor use by HIV and inhibition of HIV infection by chemokine receptor ligands”, Immunol Rev, 177:112-126, 2000.
Simmons et al., “Potent Inhibition of HIV-1 Infectivity in Macrophages and Lymphocytes by a Novel CCR5 Antagonist”, Science, 276:276-279, 1997.
Skelton et al., “Proton NMR Assignments and Solution Conformation of RANTES, a Chemokine of the C—C Type”, Biochemistry, 34:5329-5342, 1995.
Staunton et al., “A cell adhesion molecule, ICAM-1, is the major surface receptor for rhinoviruses”, Cell, 56:849-853, 1989.
Stellato et al., “Expression of the C—C chemokine receptor CCR3 in human airway epithelial cells”, J. Immunol., 166:1457-1461, 2001.
Tatusova and Madden, “BLAST 2 Sequences, a New Tool for Comparing Protein and Nucleotide Sequences”, FEMS Microbiol. Lett., 174(2):247-50, 1999.
Tekkanat et al., “RANTES (CCL5) production during primary respiratory syncytial virus infection exacerbates airway disease”, Eur J Immunol, 32:3276-3284, 2002.
Thomas et al., “Respiratory syncytial virus-induced RANTES production from human bronchial epithelial cells is dependent on nuclear factor-kappa B nuclear binding and is inhibited by adenovirus-mediated expression of inhibitor of kappa B alpha”, J. Immunol., 161:1007-1016, 1998.
Torrence et al., “The quest for an efficacious antiviral for respiratory syncytial virus”, Antivir Chem Chemother., 13:325-344, 2002.
Tripp et al., “CX3C chemokine mimicry by respiratory syncytial virus G glycoprotein”, Nat Immunol, 2:732-738, 2001.
Tyndall and Fairlie, J. Mol. Recognition, 12:363-370, 1999.
Walsh et al., “Monoclonal antibodies to respiratory syncytial virus proteins: identification of the fusion protein”, J Virol, 47:171-177, 1983.
Welliver et al., “Beta-chemokines, but neither T helper type 1 nor T helper type 2 cytokines, correlate with severity of illness during respiratory syncytial virus infection”, Pediatr Infect Dis J, 21:457-461, 2002.
Whitehead et al., “Recombinant Respiratory Syncytial Virus Bearing a Deletion of either the NS2 or SH Gene Is Attenuated in Chimpanzees”, J. Virol, 73: 3438-3442, 1999.
Wilken et al., “Total Chemical Synthesis and High-Resolution Crystal Structure of the Potent anti-HIV Protein AOP-RANTES”, Chemistry & Biology, 6:43-51, 1999.
Wilkens et al., “Protein Identification and Analysis Tools in the ExPASy Server”, 2-D Proteome Analysis Protocols (1998). Editor A. J. Link. Humana Press, New Jersey.
Wright et al., “Genetic studies of respiratory syncytial virus temperature-sensitive mutants”, Arch Gesamte Virusforsch, 41:238-247, 1973.
Zhang et al., “Expression of respiratory syncytial virus-induced chemokine gene networks in lower airway epithelial cells revealed by cDNA microarrays”. J Virol, 75:9044-9058, 2001.

What is claimed is:

1. An antiviral composition comprising a CCL5 polypeptide, wherein the CCL5 polypeptide inhibits infection by a virus of the Family Paramyxoviridae (paramyxovirus) in a mammalian subject.

2. The composition of claim 1, wherein the paramyxovirus is a respiratory syncytial virus (RSV).

3. The composition of claim 2, wherein the CCL5 polypeptide inhibits RSV infection by blocking the interaction between an RSV fusion (F) protein and a mammalian epithelial cell.

4. The composition of claim 1, wherein the CCL5 polypeptide is a synthetic CCL5 polypeptide or a recombinantly expressed CCL5 polypeptide.

5. The composition of claim 4, wherein the CCL5 polypeptide is biologically inactive as a chemokine in a mammalian subject.

6. The composition of claim 1, wherein the mammalian subject is a human.

7. The composition of claim 1, wherein the mammalian subject is a domesticated non-human mammal selected from the group consisting of a cow, a horse, a pig, a dog, a cat, a goat and a sheep.

8. The composition of claim 1, wherein the CCL5 polypeptide comprises an amino acid sequence of SEQ ID NO:1.

9. The composition of claim 1, wherein the CCL5 polypeptide is an NH₂-terminus modified CCL5 polypeptide.

10. The composition of claim 9, wherein the NH₂-terminus modified CCL5 polypeptide is selected from the group consisting of an aminooxypentane-CCL5 (AOP-CCL5), a Met-CCL5, a N^{α}-nonanoyl-CCL5 (NNY-CCL5), a Δ1-2 truncated CCL5 and a Δ1-8 truncated CCL5.

11. The composition of claim 1, further comprising one or more CCL5 peptide fragments, wherein the fragments comprise about 10 to 20 contiguous amino acids of the CCL5 polypeptide of SEQ ID NO:1.

12. The composition of claim 11, wherein the one or more CCL5 peptide fragments are selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18.

13. The composition of claim 12, wherein the CCL5 peptide fragment comprises an amino acid sequence of SEQ ID NO:2.

14. The composition of claim 13, wherein the peptide fragment of SEQ ID NO:2 is further defined as an NH₂-terminal peptide of SEQ ID NO:1.

15. The composition of claim 1, wherein the CCL5 polypeptide is further defined as a human CCL5 polypeptide.

16. The composition of claim 1, further comprising a peptide mimetic of the NH₂-terminus of the CCL5 polypeptide of SEQ ID NO:1.

17. The composition of claim 16, wherein the peptide mimetic of the NH₂-terminus of the CCL5 polypeptide is a retroinverted CCL5 polypeptide comprising an amino acid sequence of SEQ ID NO:19, SEQ ID NO:20 or SEQ ID NO:21.

18. The composition of claim 1, further comprising an organic molecule which binds a CCR3 chemokine receptor.

19. The composition of clam 18, wherein the organic molecule is a CCR3 receptor antagonist.

20. The composition of claim 19, wherein the organic molecule comprises one or more chemical structures of formula I, II or III.

21. The composition of claim 1, wherein the composition is administered to a mammalian subject by intranasal administration or parenteral administration.

22. The composition of claim 1, further comprising an organic molecule which is a CCR1 antagonist or a CCR5 antagonist.

23. A recombinant expression vector comprising a polynucleotide sequence encoding the CCL5 polypeptide of claim 1.

24. A host cell transfected, transformed or infected with the vector of claim 23.

25. An antiviral composition comprising an NH₂-terminal peptide fragment of a CCL5 polypeptide, wherein the fragment comprises about 10 to 20 contiguous amino acids of the NH₂-terminus of a CCL5 polypeptide, wherein the fragment inhibits infection by a virus of the Family Paramyxoviridae (paramyxovirus) in a mammalian subject.

26. The composition of claim 25, wherein the paramyxovirus is RSV.

27. The composition of claim 25, wherein the CCL5 polypeptide comprises an amino acid sequence of SEQ ID NO:1.

28. The composition of claim 27, wherein the NH₂-terminal peptide fragment comprises an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18.

29. The composition of claim 28, wherein the NH₂-terminal peptide fragment comprises an amino acid sequence of SEQ ID NO:2.

30. The composition of claim 25, wherein the composition is biologically inactive as a chemokine in a mammalian subject.

31. The composition of claim 25, wherein the composition is administered to a mammalian subject by intranasal administration or parenteral administration.

32. The composition of claim 25, wherein the NH₂-terminal CCL5 peptide fragment inhibits RSV infection by blocking the interaction between an RSV fusion (F) protein and a mammalian epithelial cell.

33. The composition of claim 25, further comprising one or more NH₂-terminus modified CCL5 polypeptides selected from the group consisting of an aminooxypentane-CCL5 (AOP-CCL5), a Met-CCL5, a N^{α}-nonanoyl-CCL5 (NNY-CCL5), a Δ1-2 truncated CCL5 and a Δ1-8 truncated CCL5.

34. The composition of claim 25, further comprising a peptide mimetic of the NH₂-terminus of the CCL5 polypeptide of SEQ ID NO:1.

35. The composition of claim 34, wherein the peptide mimetic of the NH₂-terminus of the CCL5 polypeptide is a retroinverted CCL5 polypeptide comprising an amino acid sequence of SEQ ID NO:19, SEQ ID NO:20 or SEQ ID NO:21.

36. The composition of claim 25, further comprising an organic molecule which is an antagonist of a CCR1 receptor, a CCR3 receptor or a CCR5 receptor.

37. A recombinant expression vector comprising a polynucleotide sequence encoding the NH₂-terminal CCL5 peptide fragment of claim 25.

38. A host cell transfected, transformed or infected with the vector of claim 37.

39. An organic small molecule mimetic which is designed by computer based molecular modeling using the atomic X, Y, Z coordinates of the first fifteen CCL5 NH₂-terminal amino acids of SEQ ID NO:1, wherein the X, Y, Z, coordinates are found in a Brookhaven Protein Data Bank file selected from the group consisting of 1RTN, 1RTO, 1 EQT and 1B3A.

40. An antiviral composition comprising an organic molecule of claim 39.

41. A peptide mimetic of the NH₂-terminus of a CCL5 polypeptide, wherein the peptide mimetic inhibits infection by a virus of the Family Paramyxoviridae (paramyxovirus) in a mammalian subject.

42. The peptide mimetic of claim 41, wherein the mimetic is designed by computer based molecular modeling using the atomic X, Y, Z coordinates of the first fifteen CCL5 NH₂-terminal amino acids of SEQ ID NO:1, wherein the X, Y, Z, coordinates are comprised in a Brookhaven Protein Data Bank file selected from the group consisting of 1RTN, 1RTO, 1EQT and 1B3A.

43. The of peptide mimetic of claim 41, wherein the mimetic is a reverse turn mimetic.

44. The peptide mimetic of claim 43, wherein the reverse turn mimetic is a β-turn mimetic, a monocyclic β-turn mimetic, a bicyclic β-turn mimetic, a γ-turn mimetic or a monocyclic γ-turn mimetic.

45. An antiviral composition comprising the peptide mimetic of claim 41.

46. A method for preventing or inhibiting infection by a virus of the Family Paramyxoviridae (paramyxovirus) in a mammalian host, the method comprising administering to the host a pharmaceutically effective amount of the composition of claim 1.

47. A method for preventing or inhibiting paramyxovirus infection in a mammalian host, the method comprising administering to the host a pharmaceutically effective amount of the composition of claim 25.

48. A method for preventing or inhibiting paramyxovirus infection in a mammalian host, the method comprising administering to the host a pharmaceutically effective amount of the composition of claim 39.

49. A method for preventing or inhibiting infection by a virus of the Family Paramyxoviridae (paramyxovirus) in a mammalian host, the method comprising administering to the host a pharmaceutically effective amount of the composition of claim 41.


SEQUENCE LISTING (22 sequences)

SEQ ID NO: 1 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 2 - protein, 15 aa, Homo sapiens
SPYSSDTTPCCFAYI

SEQ ID NO: 3 - protein, 15 aa, Homo sapiens
TPCCFAYIARPLPRA

SEQ ID NO: 4 - protein, 15 aa, Homo sapiens
IARPLPRAHIKEYFY

SEQ ID NO: 5 - protein, 15 aa, Homo sapiens
AHIKEYFYTSGKCSN

SEQ ID NO: 6 - protein, 15 aa, Homo sapiens
YTSGKCSNPAVVFVT

SEQ ID NO: 7 - protein, 14 aa, Homo sapiens
PAVVFVTRKNRQVC

SEQ ID NO: 8 - protein, 15 aa, Homo sapiens
TRKNRQVCANPEKKW

SEQ ID NO: 9 - protein, 15 aa, Homo sapiens
CANPEKKWVREYINS

SEQ ID NO: 10 - protein, 15 aa, Homo sapiens
EKKWVREYINSLEMS

SEQ ID NO: 11 - protein, 12 aa, Homo sapiens
TTPCCFAYIARP

SEQ ID NO: 12 - protein, 12 aa, Homo sapiens
PCCFAYIARPLP

SEQ ID NO: 13 - protein, 12 aa, Homo sapiens
CFAYIARPLPRA

SEQ ID NO: 14 - protein, 12 aa, Homo sapiens
HIKEYFYTSGKC

SEQ ID NO: 15 - protein, 12 aa, Homo sapiens
SGKCSNPAVVFV

SEQ ID NO: 16 - protein, 19 aa, Homo sapiens
CFAYIARPLPRAHIKEYFY

SEQ ID NO: 17 - protein, 24 aa, Homo sapiens
CFAYIARPLPRAHIKEYFYTSGKC

SEQ ID NO: 18 - protein, 34 aa, Homo sapiens
SPYSSDTTPCCFAYIARPLPRAHIKEYFYTSGKC

SEQ ID NO: 19 - protein, 19 aa, Homo sapiens
YFYEKIHARPLPRAIYAFC

SEQ ID NO: 20 - protein, 34 aa, Homo sapiens
CKGSTYFYEKIHARPLRPAIYAFCCPTTDSSYPS

SEQ ID NO: 21 - protein, 15 aa, Homo sapiens
IYAFCCPTTDSSYPS

SEQ ID NO: 22 - protein, 69 aa, Homo sapiens
SLAADTPTACCFSYTSRQIPQNFIAAYFETSSQCSKPGVIFLTKRSRQVCADPSEEWVQKYVSDLELSA